(19) United States
(12) Patent Application Publication
(10) Pub. No.: US 2019/0114722 A1
(43) Pub. Date: Apr. 18, 2019

(54) INFORMATION PROCESSING SYSTEM AND INFORMATION PROCESSING APPARATUS

(71) Applicant: FUJITSU LIMITED, Kawasaki-shi (JP)

(72) Inventors: Yuri Shimada, Yokohama (JP); Hiroyuki ANDO, Sayama (JP); Akito YAMAZAKI, Kawasaki (JP); Hiroaki Kanto, Yokohama (JP)

(73) Assignee: FUJITSU LIMITED, Kawasaki-shi, (JP)

(21) Appl. No.: 16/142,032

(22) Filed: Sep. 26, 2018

(30) Foreign Application Priority Data
Oct. 13, 2017 (JP) 2017-199730

(51) Int. Cl. G06Q 40/00; G16H 10/60

(52) U.S. Cl. CPC G06Q 40/123; G16H 10/60

(57) ABSTRACT

An information processing system includes, a first information processing apparatus that stores medical information associated with identification information, a second information processing apparatus that stores personal information associated with identification information, and a third information processing apparatus that transmits, to the first information processing apparatus, first identification information that identifies medical information usable for specific application, receives medical information corresponding to the first identification information from the first information processing apparatus, transmits, to the second information processing apparatus, second identification information that identifies personal information usable for the specific application, receives personal information corresponding to the second identification information from the second information processing apparatus, and generates application information usable for the specific application, based on the medical information received from the first information processing apparatus and the personal information received from the second information processing apparatus.

### CROSS-REFERENCE TO RELATED APPLICATION

This application is based upon and claims the benefit of priority of the prior Japanese Patent Application No. 2017-199730, filed on Oct. 13, 2017, the entire contents of which are incorporated herein by reference.

### FIELD

The embodiments discussed herein are related to an information processing system and an information processing apparatus.

### BACKGROUND

Recently, a government has planned to promote the utilization of medical data in response to a growing demand for the utilization of data. According to the plan, the government collects medical information in hospitals and utilizes the collected medical information for big data analysis or personalized services to promote data utilization in domestic medical fields.

Personal data stores (PDS) have attracted attention due to the growing demand for the utilization of personal information in business. PDS is a technology that enables individuals to accumulate and manage all personal information about themselves and enables the personal information to be utilized for a service of a supplier to which the individual has granted access rights to the personal information.

In a case in which an applicant is eligible for a medical expense deduction by way of a final tax return, the applicant keeps data such as receipts of medical expenses to be deducted. The applicant submits data by preparing a handwritten declaration or by inputting data to the declaration by using a personal computer (PC). The medical expenses to be deducted range from hospital treatment costs, hospital room costs, transportation costs between home and a hospital, to nursing care expenses, and the like, and therefore, collection and input of data become a burden.

A self-medication tax system has been introduced since FY 2017, and the applicant may receive a deduction through a self-medication tax system in addition to the ordinary medical expense deduction. The self-medication tax system is a tax system in which the applicant may receive an income deduction when the applicant has purchased commercially available drugs that are deduction targets at a specific amount or more in a drug store or the like. As a result, the applicant selects one of the ordinary medical expense deduction or the self-medication tax system deduction for filing a final tax return.

A system to apply for a medical expense deduction or the like, a system that collects information while taking privacy into consideration, and the like are also known.

For example, Japanese Laid-open Patent Publication No. 2013-257618, Japanese Laid-open Patent Publication No. 2002-15087, and Japanese Laid-open Patent Publication No. 2016-152024 disclose the related art.

### SUMMARY

According to an aspect of the embodiments, an information processing system includes a first information processing apparatus that stores medical information associated with identification information, a second information processing apparatus that stores personal information associated with identification information, and a third information processing apparatus that transmits, to the first information processing apparatus, first identification information that identifies medical information usable for specific application, receives medical information corresponding to the first identification information from the first information processing apparatus, transmits, to the second information processing apparatus, second identification information that identifies personal information usable for the specific application, receives personal information corresponding to the second identification information from the second information processing apparatus, and generates application information usable for the specific application, based on the medical information received from the first information processing apparatus and the personal information received from the second information processing apparatus.

The object and advantages of the invention will be realized and attained by means of the elements and combinations particularly pointed out in the claims. It is to be understood that both the foregoing general description and the following detailed description are exemplary and explanatory and are not restrictive of the invention.

### BRIEF DESCRIPTION OF DRAWINGS

FIG. 1 is a configuration diagram of an information processing system;

FIG. 2 is a flowchart of information processing;

FIG. 3 is a configuration diagram illustrating a specific example of the information processing system;

FIG. 4 is a diagram illustrating collection processing of collecting information on a transportation expense;

FIG. 5 is a diagram illustrating collection processing of collecting medicine purchase history information;

FIG. 6 is a diagram illustrating merging processing;

FIG. 7 is a diagram illustrating merged information;

FIG. 8 is a diagram illustrating deletion processing;

FIG. 9 is a diagram illustrating management information;

FIG. 10 is a diagram illustrating data for a medical expense deduction;

FIG. 11 is a diagram illustrating data for a deduction through a self-medication tax system;

FIG. 12 is a diagram illustrating an analysis result;

FIG. 13 is a diagram illustrating a registration sequence of registering personal information;

FIG. 14 is a diagram illustrating a registration sequence of registering management information;

FIGS. 15A-1 and 15A-2 are diagrams illustrating a collection sequence of collecting medical information;

FIGS. 15B-1 and 15B-2 are diagrams illustrating the collection sequence of collecting the medical information;

FIGS. 16A-1 and 16A-2 are diagrams illustrating a collection sequence of collecting personal information;

FIGS. 16B-1 and 16B-2 are diagrams illustrating the collection sequence of collecting the personal information;

FIG. 16C is a diagram illustrating the collection sequence of collecting the personal information;

FIGS. 17A-1 and 17A-2 are diagrams illustrating generation sequence of generating application information;

FIGS. 17B-1 and 17B-2 are diagrams illustrating the generation sequence of generating the application information; and

FIG. 18 is a hardware configuration diagram of the information processing apparatus.

### DESCRIPTION OF EMBODIMENTS

The introduction of the self-medication tax system enables the applicant to select one of the ordinary medical expense deduction or the self-medication tax system deduction. Therefore, the number of types of medical information and personal information increases due to the increase in the number of choices, and a task of collecting such pieces of information is further complicated.

Such a problem is not limited to the final tax return and also occurs in other applications such as insurance claims, high-cost medical expenses refunds, and the like.

Details of embodiments of the technology discussed herein are described with reference to the drawings. As described above, due to the introduction of the self-medication tax system, the task of collecting medical information and personal information that may be used for the final tax return and creating a declaration based on the information is further complicated.

For example, to be eligible for a deduction by using the self-medication tax system, the receipts of commercially-available medicine may be kept over a long period of time, which takes time and effort. The amount of money of the medical expense deduction and the self-medication tax system deduction differs depending on the applicant, and therefore, it is difficult to determine which deduction is suitable for individual applicants. In addition, a user who usually does not file a final tax return may not be aware that they are eligible for the medical expense deduction or the self-medication tax system deduction.

Medical information collected from hospitals according to the above-described government plan includes information on medical expenses that may be used for the final tax return but does not include the price of medicine purchased by the applicant without going through a hospital. Therefore, it is difficult to estimate the monetary amount of deductions by the self-medication tax system based only on the medical information collected from hospitals.

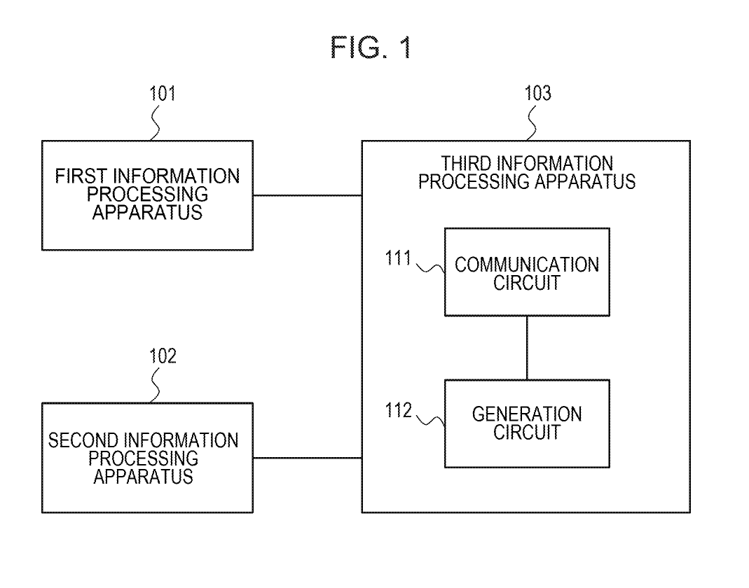

FIG. 1 illustrates a configuration diagram of an information processing system according to an embodiment. The information processing system illustrated in FIG. 1 includes a first information processing apparatus 101, a second information processing apparatus 102, and a third information processing apparatus 103. The first information processing apparatus 101 stores medical information associated with identification information, and the second information processing apparatus 102 stores personal information associated with identification information. The third information processing apparatus 103 includes a communication circuit 111 and a generation circuit 112.

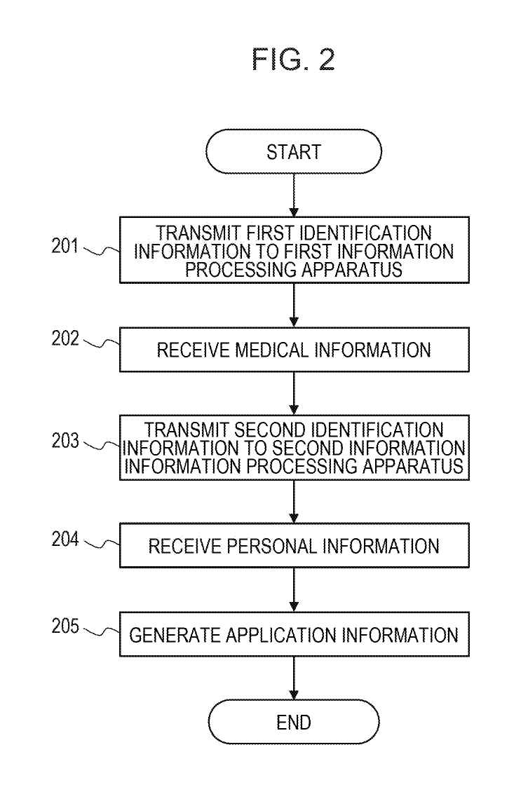

FIG. 2 is a flowchart illustrating an example of information processing executed by the third information processing apparatus 103 in FIG. 1. First, the communication circuit 111 transmits first identification information that identifies medical information usable for a specific application to the first information processing apparatus 101 (step 201) and receives medical information corresponding to the first identification information from the first information processing apparatus 101 (step 202). Next, the communication circuit 111 transmits second identification information that identifies personal information usable for the specific application to the second information processing apparatus 102 (step 203) and receives personal information corresponding to the second identification information from the second information processing apparatus 102 (step 204).

The generation circuit 112 generates application information usable for the specific application based on the medical information received from the first information processing apparatus 101 and the personal information received from the second information processing apparatus 102 (step 205).

The order of the processing in steps 201 to 204 may differs from the order illustrated in FIG. 2. For example, the communication circuit 111 may execute the processing in steps 203 and 204 prior to the processing in steps 201 and 202.

In the information processing system illustrated in FIG. 1, medical information and personal information that may be used for applications related to medical fields are collected.

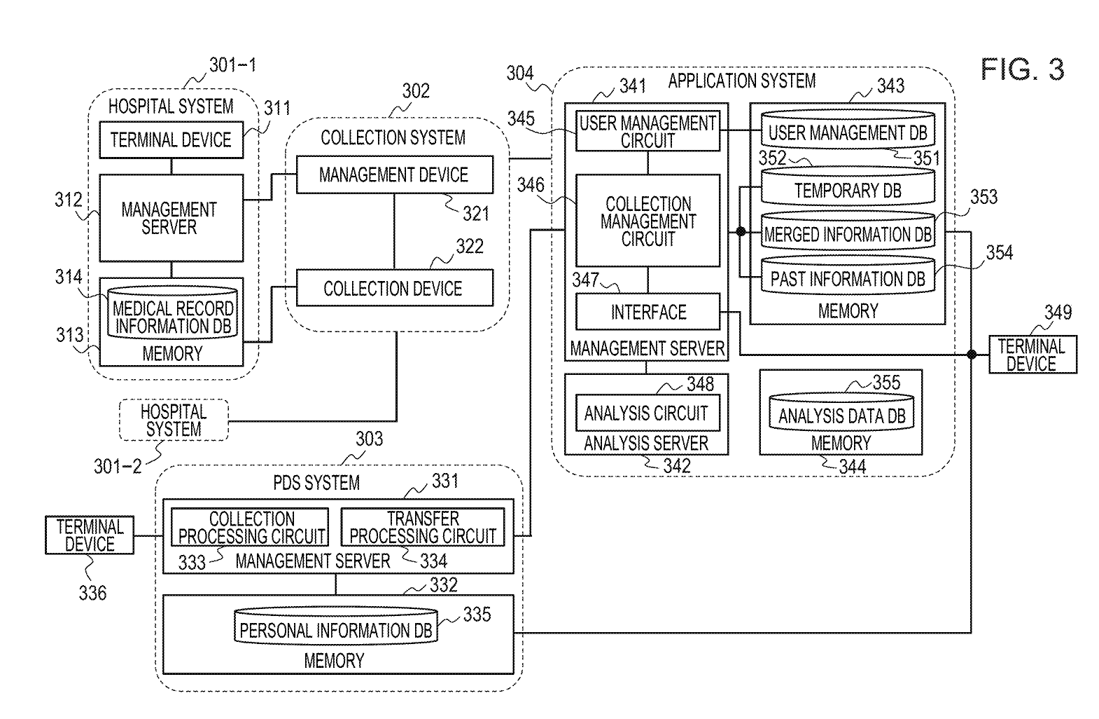

FIG. 3 illustrates a specific example of the information processing system in FIG. 1. The information processing system illustrated in FIG. 3 includes a hospital system 301-1 of a hospital A, a hospital system 301-2 of a hospital B, a collection system 302, a PDS system 303, and an application system 304 for a final tax return.

The number of hospital systems is not limited to two, and when the number of hospitals is three or more, there is a corresponding number of hospital systems. For example, such hospitals may be distributed throughout the country or may exist in a specific region. The collection system 302 is used when a data collection organization such as the government collects medical information from hospital systems.

The hospital system 301-1 includes a terminal device 311, a management server 312, and a memory 313, and the memory 313 includes a medical record information database (DB) 314.

The terminal device 311 enables a doctor or a clerk to input medical information including a cost item and a medical expense of a patient as to an electronic medical record, and transmits the electronic medical record to the management server 312. The management server 312 stores the electronic medical record received from the terminal device 311 in the medical record information DB 314. The memory 313 transmits the electronic medical record in the medical record information DB 314 to the collection system 302 in response to an instruction from the management server 312. The configuration and operation of the hospital system 301-2 are similar to those of the hospital system 301-1.

The collection system 302 includes a management device 321 and a collection device 322. The management device 321 and the collection device 322 correspond to the first information processing apparatus 101 illustrated in FIG. 1. The collection device 322 stores an electronic medical record that has been received from the memory 313, and the management device 321 transmits medical information included in an electronic medical record of a specific patient to the application system 304 in response to a request from the application system 304.

The PDS system 303 includes a management server 331 and a memory 332. The management server 331 and the memory 332 correspond to the second information processing apparatus 102 illustrated in FIG. 1. The management server 331 includes a collection processing circuit 333 and a transfer processing circuit 334, and the memory 332 includes a personal information DB 335. A terminal device 336 transmits, to the management server 331 in response to an operation of the user, personal information such as a purchase history of items that have been purchased by the user and a transportation expense that has been paid by the user and the collection processing circuit 333 stores personal information that has been received from the terminal device 336 in the personal information DB 335. The transfer processing circuit 334 transmits personal information of a specific user to the application system 304 in response to a request from the application system 304.

The application system 304 includes a management server 341, an analysis server 342, a memory 343, and a memory 344 and provides the user with a final tax return service to generate application information for the final tax return. The management server 341 and the analysis server 342 correspond to the third information processing apparatus 103 illustrated in FIG. 1.

The management server 341 includes a user management circuit 345, a collection management circuit 346, and an interface 347, and the analysis server 342 includes an analysis circuit 348. The collection management circuit 346 and the analysis circuit 348 correspond to the generation circuit 112 in FIG. 1. The memory 343 includes a user management DB 351, a temporary DB 352, a merged information DB 353, and a past information DB 354, and the memory 344 includes an analysis data DB 355.

A terminal device 349 transmits management information of a user who is an applicant for the final tax return to the management server 341 in response to an operation of the user, and the interface 347 outputs the management information that has been received from the terminal device 349 to the user management circuit 345. The user management circuit 345 transfers the management information to the memory 343, and the memory 343 stores the management information that has been received from the user management circuit 345 in the user management DB 351.

The management information of the user includes login information of the final tax return service, authentication information of the collection system 302, authentication information of the PDS system 303, and family information. The login information of the final tax return service includes user identification information (user ID) of the final tax return service, which has been applied to the user.

The authentication information of the collection system 302 includes a user ID of the collection system 302 which has been applied to the user, and the authentication information of the PDS system 303 includes a user ID of the PDS system 303 which has been applied to the user. The user ID in the collection system 302 is used to identify an electronic medical record stored in the collection device 322, and the user ID in the PDS system 303 is used to identify personal information stored in the personal information DB 335.

The user management circuit 345 obtains management information of the user who is eligible for the final tax return service from the user management DB 351 and outputs the management information to the collection management circuit 346.

The collection management circuit 346 transmits the user ID in the collection system 302 as first identification information to the collection system 302 and requests the collection system 302 to provide medical information of the user. The collection system 302 transmits medical information of a patient corresponding to the received user ID to the memory 343, and the memory 343 stores the received medical information in the temporary DB 352. The collection management circuit 346 converts the medical information in the temporary DB 352 into a data format usable for a declaration of the final tax return and stores the converted medical information in the merged information DB 353.

The collection management circuit 346 transmits the user ID in the PDS system 303 as second identification information to the PDS system 303 and requests the PDS system 303 to provide personal information of the user. The PDS system 303 transmits personal information of the user corresponding to the received user ID to the memory 343, and the memory 343 stores the received personal information in the temporary DB 352.

Next, the collection management circuit 346 converts the personal information in the temporary DB 352 into the data format usable for the declaration of the final tax return and compares the personal information having the converted data format with the medical information stored in the merged information DB 353. The collection management circuit 346 generates merged information by merging the medical information with the personal information while detecting duplicate information in the medical information and the personal information and stores the merged information in the merged information DB 353.

Next, the collection management circuit 346 compares the merged information in the merged information DB 353 with analysis data stored in the past information DB 354, which has been generated in the past, and deletes information that duplicates the analysis data in the past information DB 354 from the merged information DB 353.

The analysis circuit 348 of the analysis server 342 analyzes the merged information in the merged information DB 353 to generate analysis data and stores the generated analysis data in the analysis data DB 355. The analysis data includes merged information that is an analysis target and the accompanying analysis result. The management server 341 obtains the analysis data from the analysis server 342, and the interface 347 transmits the analysis data as application information to the terminal device 349. The analysis data may include a declaration for the final tax return.

In the information processing system illustrated in FIG. 3, for example, the management server 341 collects medical information of the user from the collection system 302 in the following procedures.

(P1) The user who utilizes the final tax return service inputs management information of the user to the terminal device 349 through a user interface such as a browser. The terminal device 349 transmits the input management information to the management server 341, and the user management circuit 345 stores the received management information in the user management DB 351. As a result, the management information of the user is registered in the user management DB 351.
(P2) The collection management circuit 346 obtains, from the user management DB 351 through the user management circuit 345, management information of the user whose medical information is to be collected. As a result, collection of the medical information is started. As the collection start timing, for example, the following timings are assumed.
(a) Timing at which a request from the user is received
(b) Periodically (every month, every week, or the like)
(c) During a time from the beginning of the year to the final tax return declaration start
(P3) The collection management circuit 346 transmits the user ID in the collection system 302 to the collection system 302 through a plug-in for the collection system 302 and requests the collection system 302 to provide medical information of the user.
(P4) The management device 321 obtains an electronic medical record corresponding to the user ID from among electronic medical records stored in the collection device 322 in response to the request from the collection management circuit 346 and transmits medical information included in the obtained electronic medical record to the memory 343. The memory 343 stores the received medical information in the temporary DB 352.
(P5) The collection management circuit 346 converts the medical information in the temporary DB 352 into the data format usable for the declaration of the final tax return, associates the converted medical information with the user ID for the final tax return service, and stores the medical information and the user ID in the merged information DB 353.
(P6) The collection management circuit 346 deletes the medical information in the temporary DB 352.

Next, the management server 341 collects personal information of the user from the PDS system 303, for example, in the following procedures.

(P11) The user purchases medicine in a drug store and inputs, to the terminal device 336, medicine purchase history information including the price of the purchased medicine. The terminal device 336 transmits the input information to the management server 331, and the collection processing circuit 333 stores the received personal information in the personal information DB 335.

When the terminal device 336 is a terminal device of a drug store, a salesperson of the drug store inputs medicine purchase history information to the terminal device 336 on behalf of the user.

(P12) The collection management circuit 346 obtains, from the user management DB 351 through the user management circuit 345, management information of the user whose personal information is to be collected. The user whose personal information is to be collected is the same person whose medical information has been collected.
(P13) The collection management circuit 346 transmits the user ID in the PDS system 303 to the PDS system 303 and requests the PDS system 303 to provide the personal information of the user. At this time, the collection management circuit 346 transmits a request in a format that conforms to a data providing interface of the PDS system 303 to the management server 331 through the plug-in for the PDS system 303. For example, in a case of a database of PostgreSQL, a command of PostgreSQL is used as the request.

For the final tax return, a declaration based on the undeclared information up to the past five years may be submitted. Therefore, the collection management circuit 346 requests the PDS system 303 to provide personal information up to the past five years in order to check personal information before the current year by assuming a case in which the user has added the past personal information to the PDS system 303. However, when the PDS system 303 provides only personal information updated after personal information has been previously requested, the collection management circuit 346 may request the PDS system 303 to provide personal information for the past one year.

(P14) The transfer processing circuit 334 obtains personal information corresponding to the user ID from among pieces of personal information stored in the personal information DB 335 in response to the request from the collection management circuit 346 and transmits the obtained personal information to the memory 343. The memory 343 stores the received personal information in the temporary DB 352.
(P15) The collection management circuit 346 converts the data format of the personal information in the temporary DB 352 from the data format of the PDS system 303 to the data format usable for the declaration of the final tax return.
(P16) The collection management circuit 346 checks for the presence or absence of duplicate information by comparing items such as date and time, hospital name, and amount of money which constitute medical information stored in the merged information DB 353 with the personal information in the temporary DB 352. The collection management circuit 346 extracts only information on the personal information, which is not a duplicate of the medical information, in the temporary DB 352 and stores the extracted information in the merged information DB 353. As a result, the collection management circuit 346 merges the personal information in the temporary DB 352 with the medical information in the merged information DB 353 to generate merged information.
(P17) The collection management circuit 346 checks for the presence or absence of duplicate information by comparing items such as date and time, hospital name, and amount of money, which constitute analysis data for the past five years stored in the past information DB 354 with the merged information in the merged information DB 353. The collection management circuit 346 deletes information previously stored in the past information DB 354 from the merged information DB 353.
(P18) The collection management circuit 346 deletes the personal information in the temporary DB 352.

Next, the management server 341 and the analysis server 342 generate application information and provide the user with the generated application information, for example, in the following procedures.

(P21) The analysis circuit 348 transmits an inquiry about management information of the user, which is an analysis target, to the management server 341.
(P22) The user management circuit 345 transmits the management information of the user, which is the analysis target, to the analysis circuit 348.
(P23) The analysis circuit 348 reads merged information corresponding to the user ID for the final tax return service as analysis target data from the merged information DB 353.
(P24) The analysis circuit 348 analyzes the analysis target data to generate an analysis result indicating that a deduction may be applied from among a medical expense deduction and a self-medication tax system deduction. At this time, the analysis circuit 348 combines undeclared information before the current year for each year and generates an analysis result related to the information when the final tax return may be filed.
(P25) The analysis circuit 348 associates the analysis data including the analysis target data and the analysis result with the user ID for the final tax return service and stores the analysis data and the user ID in the analysis data DB 355.
(P26) The interface 347 transmits the analysis data as application information to the terminal device 349 through e-mail or the like and requests the user to confirm the application information and provide missing medical information or personal information.
(P27) The user confirms the received application information and transmits the requested missing information to the management server 341 through a user interface such as a browser or the like when information that supports a deduction request is missing.
(P28) The collection management circuit 346 transfers the information that has been received from the terminal device 349 to the analysis server 342, and the analysis circuit 348 adds the received information to the analysis target data.
(P29) The application system 304 completes the application information by repeating the procedures P24 to P28. In the procedure P27, when information that supports a deduction request is sufficient, the user files the final tax return based on the received application information.

In the information processing system illustrated in FIG. 3, as long as the user records, in the PDS system 303, information on receipts in which the price of transportation expenses, medicine, and the like, which are deduction targets, are described, it becomes unnecessary for the user to store the physical receipts for a long time period. In addition, the application system 304 automatically collects medical information and personal information that may be used for the final tax return.

The application system 304 may collect medical information and personal information in accordance with a declaration time period of the final tax return to generate application information and provide the user with the application information. As a result, even when the user is not aware that the information is an application target for the medical expense deduction or the self-medication tax system deduction, the user may reliably file the final tax return.

Duplicate information in medical information collected from the collection system 302 and personal information collected from the PDS system 303 may be detected easily by converting the medical information and the personal information into the data format usable for the declaration of the final tax return. As a result, the analysis may be performed by merging the medical information with the personal information.

The application system 304 in FIG. 3 may also be used to generate application information for other applications such as an insurance claim and a high-cost medical expense refund in addition to the final tax return. In this case, the application system 304 collects medical information and personal information that may be used for an application filed by the user from the collection system 302 and the PDS system 303, respectively.

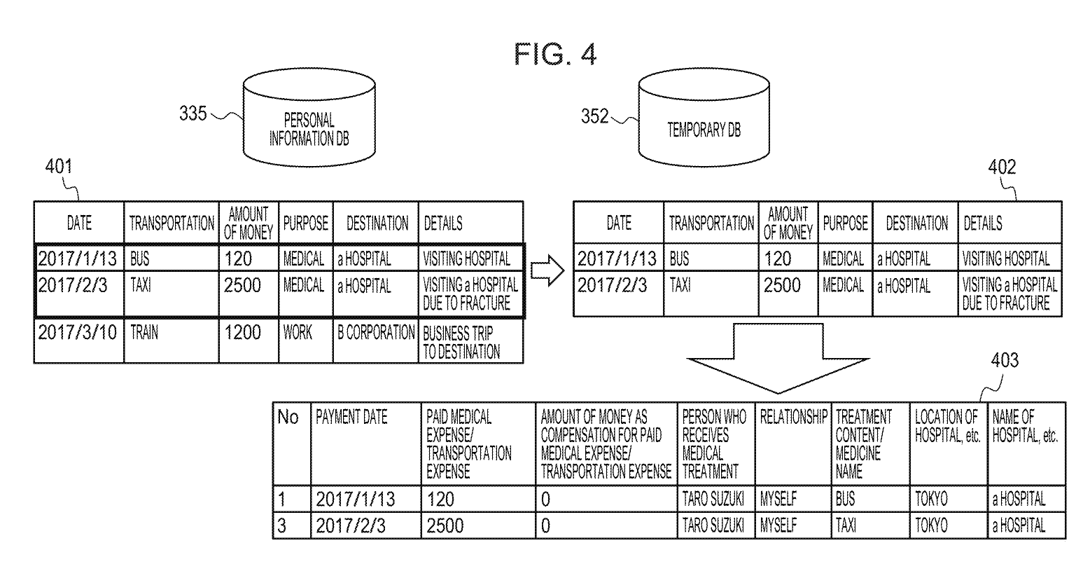

FIG. 4 illustrates an example of the collection processing in which the collection management circuit 346 collects information on a transportation expense that is a target of the medical expense deduction from the PDS system 303 in the procedures P13 to P15. The collection management circuit 346 converts items in the declaration of the final tax return into the format that conforms to the data provision interface of the PDS system 303 and transmits, to the management server 331, a request for the management server 331 to provide personal information of “transportation expense (medical-related transportation expense)”. Items of “transportation expense (medical-related transportation expense)” included in the declaration of the final tax return are as follows.

(T1) Payment date
(T2) Paid medical expense and transportation expense
(T3) Amount of money as compensation for the paid medical expense and transportation expense
(T4) People who receives medical treatment (relationship)
(T5) Treatment content/medicine name
(T6) Location of hospital or the like
(T7) Name of hospital or the like

The personal information DB 335 stores personal information 401 related to transportation expenses of the user, which is associated with the user ID in the PDS system 303. The personal information 401 includes items such as date, transportation, amount of money, purpose, destination, and details. “Date” indicates a date on which the user moves, “transportation” indicates the transportation used for the movement, “amount of money” indicated a paid transportation expense, “purpose” indicates the purpose of the movement, “destination” indicates the destination of the movement, and “details” indicate the content of the movement.

In this case, the transfer processing circuit 334 extracts, from among entries of the personal information 401, an entry in which “purpose” is “medical” and transmits the extracted entry to the collection management circuit 346. The collection management circuit 346 stores personal information 402 of the received entry in the temporary DB 352 and converts the personal information 402 into personal information 403 having the data format for the declaration.

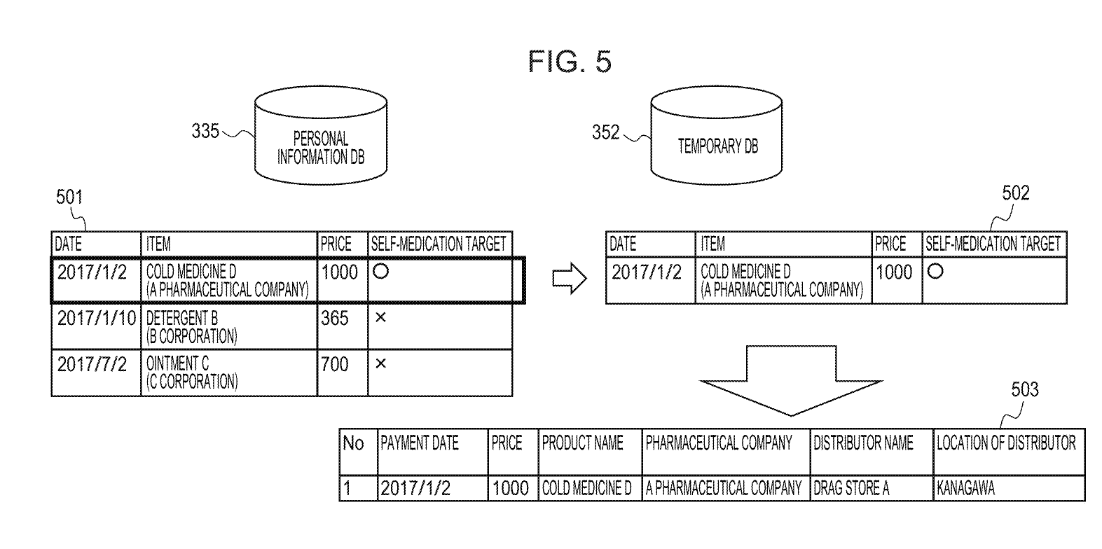

FIG. 5 illustrates an example of the collection processing in which the collection management circuit 346 collects medicine purchase history information that is a target of the self-medication tax system deduction from the PDS system 303 in the procedures P13 to P15. The collection management circuit 346 converts items of the declaration of the final tax return into the format that conforms to the data provision interface of the PDS system 303 and transmits, to the management server 331, a request for the management server 331 to provide personal information on “self-medication”. Items of “self-medication” included in the declaration of the final tax return are as follows.

(M1) Payment date

### (M2) Price

(M3) Product name
(M4) Pharmaceutical company
(M5) Distributor name
(M6) Location of the distributor

The personal information DB 335 stores personal information 501 on a medicine purchase history of the user, which is associated with the user ID in the PDS system 303. The personal information 501 includes items such as date, item, price, and self-medication target. “Date” indicates a date on which the user purchases medicine, “item” indicates a product name and a pharmaceutical company of the medicine, “price” indicates the price of the medicine, and “self-medication target” indicates whether the medicine is a deduction target by the self-medication tax system. Here, “0” indicates that the medicine is a deduction target, and “x” indicates that the medicine is not a deduction target.

In this case, the transfer processing circuit 334 extracts an entry in which the “self-medication target” is “0” from among entries of the personal information 501 and transmits the extracted entry to the collection management circuit 346. The collection management circuit 346 stores personal information 502 of the received entry in the temporary DB 352 and converts the personal information 502 into personal information 503 having the data format of the declaration.

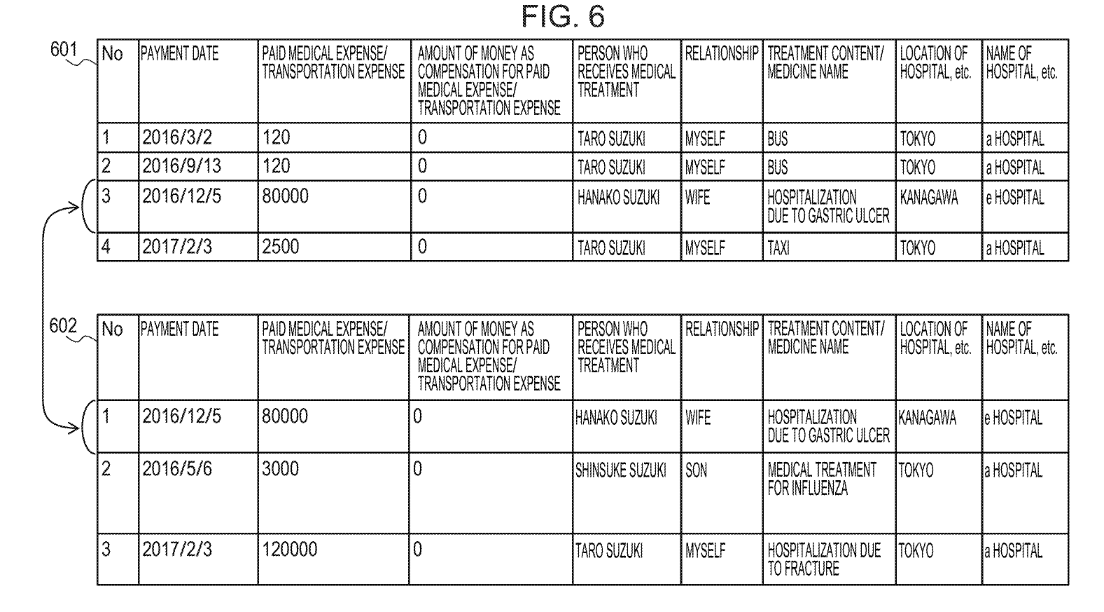

FIG. 6 illustrates an example of the merging processing executed by the collection management circuit 346 in the procedure P16. Personal information 601 corresponds to personal information collected from the PDS system 303 and stored in the temporary DB 352, and medical information 602 corresponds to medical information collected from the collection system 302 and stored in the merged information DB 353.

In this case, information on an entry of No. 3 in the personal information 601 matches information on an entry of No. 1 in the medical information 602, and the pieces of information indicate the same hospitalization history of the same patient. Therefore, the collection management circuit 346 deletes the entry of No. 3 in the personal information 601, which duplicates the entry of the medical information 602, from the personal information 601, extracts only information on the remaining entries in the personal information 601, and merges the extracted information with the medical information 602.

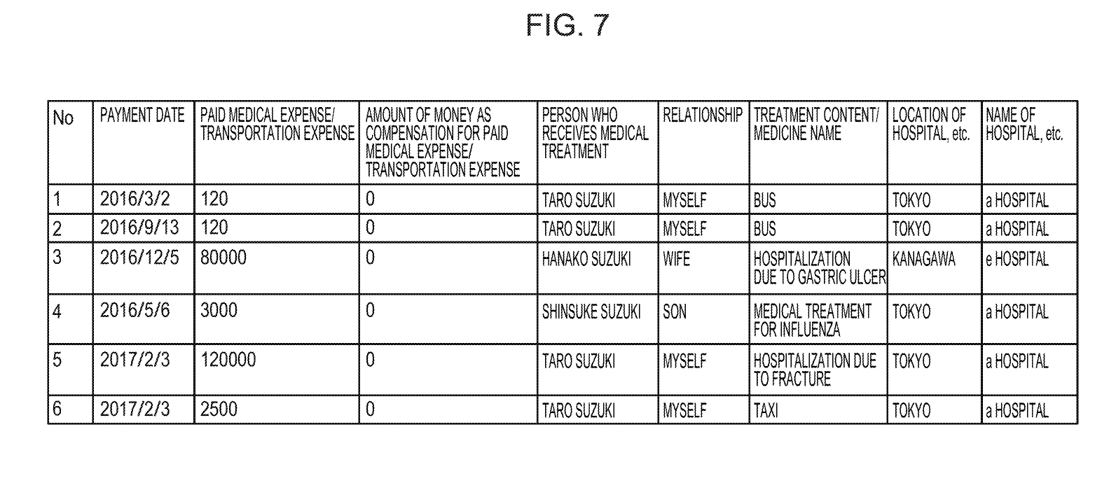

FIG. 7 illustrates an example of the merged information generated in the merging processing in FIG. 6. From among entries of the merged information in FIG. 7, entries of Nos. 3 to 5 correspond to the medical information 602 in FIG. 6, and entries of Nos. 1, 2, and 6 correspond to the personal information 601 in FIG. 6.

As described above, the medical information collected from the collection system 302 is merged with the personal information collected from the PDS system 303 to generate medical expense deduction information (merged information) that may be used when the user apply for a medical expense deduction. The generated medical expense deduction information includes a medical expense paid for a medical institution such as a hospital by the user and a transportation expense paid for a medical examination at the medical institution by the user.

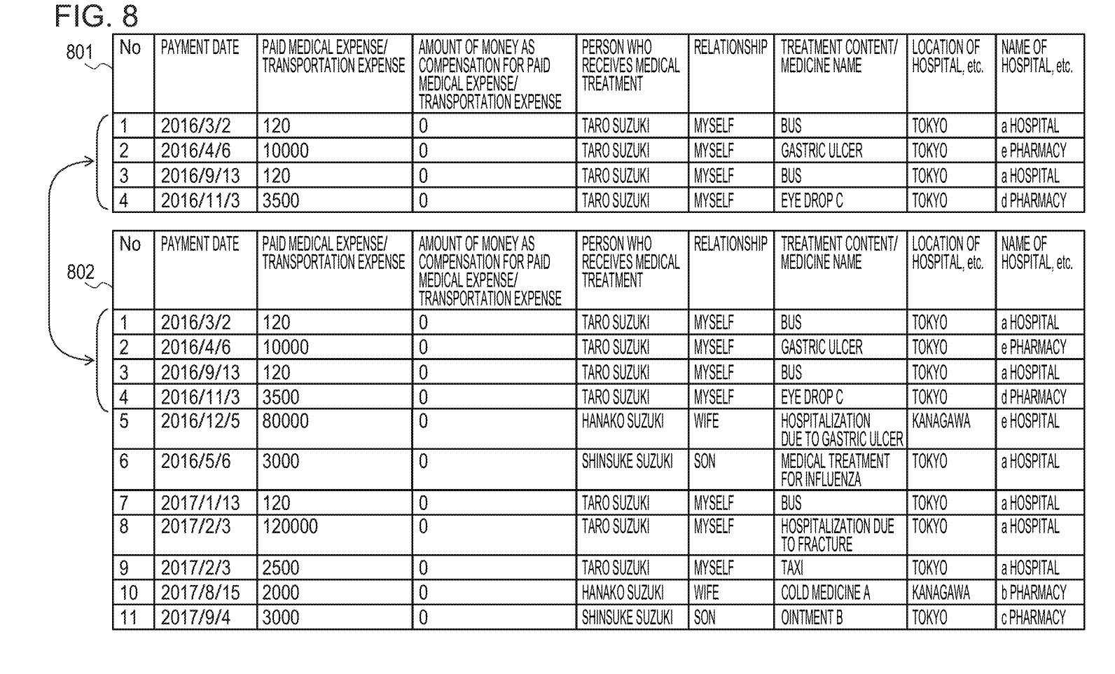

FIG. 8 illustrates the deletion processing executed by the collection management circuit 346 in the procedure P17. Merged information 801 corresponds to merged information included in analysis data for the past five years stored in the past information DB 354, and merged information 802 corresponds to merged information stored in the merged information DB 353.

In this case, information on entries of Nos. 1 to 4 in the merged information 801 matches information on entries of Nos. 1 to 4 in the merged information 802. Therefore, the collection management circuit 346 deletes the entries of Nos. 1 to 4 in the merged information 802, which duplicate the entries of the merged information 801, from the merged information 802.

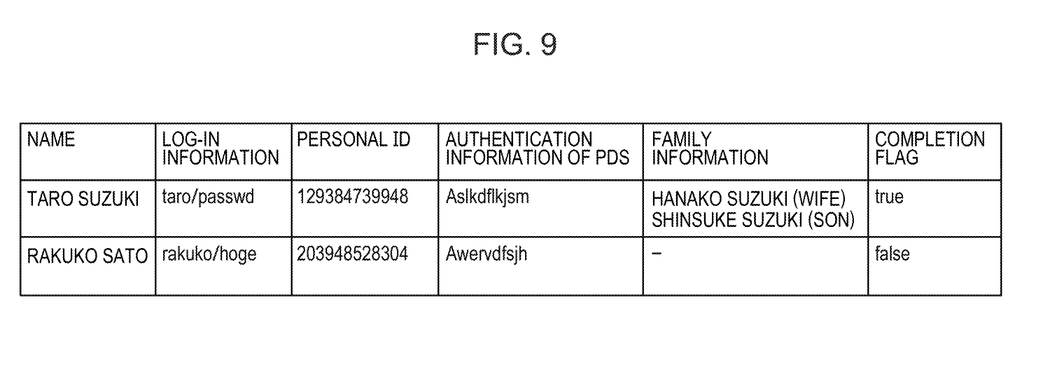

FIG. 9 illustrates an example of management information stored in the user management DB 351. The management information in FIG. 9 includes items such as name, login information, personal ID, authentication information of the PDS, family information, and completion flag.

“Name” indicates the name of the user, “login information” indicates a user ID and a password usable for the final tax return service, “personal ID” indicated a user ID usable in the collection system 302, and “authentication information of the PDS” indicates a user ID usable in the PDS system 303. “Family information” indicates information about a family (wife, child, and the like) of the user, and “completion flag” indicates whether generation of application information has been completed. Here, “true” indicates that generation of application information has been completed, and “false” indicates that generation of application information is yet to be completed.

When family information is added to the management information of the user, the application system 304 may collect medical information and personal information of the whole family of the user to generate application information of the final tax return for each household.

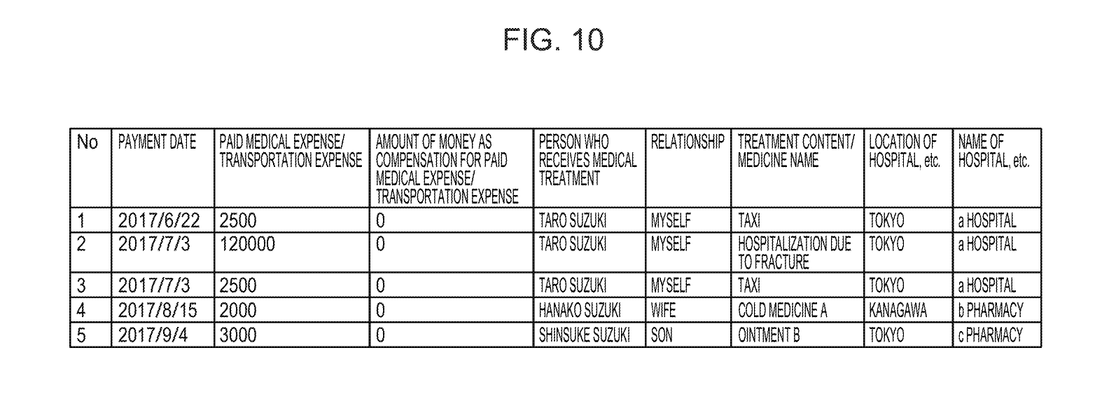

FIG. 10 illustrates an example of data for a medical expense deduction of Suzuki family, which is included in merged information stored in the merged information DB 353. Items of the data in FIG. 10 are similar to those of the personal information 403 illustrated in FIG. 4. In the management information in FIG. 9, Taro Suzuki who is a householder of Suzuki family is registered as a user of the final tax return service.

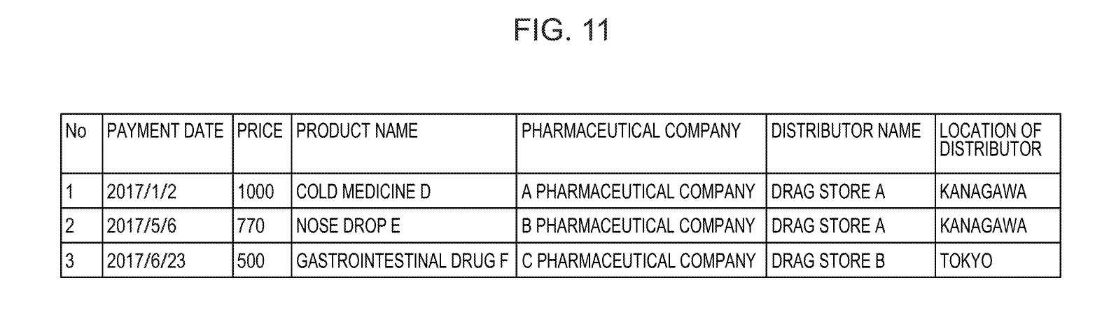

FIG. 11 illustrates an example of data for a self-medication tax system deduction of Suzuki family, which is included in merged information stored in the merged information DB 353. Items of the data in FIG. 11 are similar to those of the personal information 503 in FIG. 5.

An example of the analysis processing executed by the analysis circuit 348 of the analysis server 342 in the procedure P24 is described below. The analysis circuit 348 calculates a medical expense deduction and a self-medication tax system deduction based on the data of Suzuki family in FIGS. 10 and 11.

### (C1) Calculation of a Medical Expense Deduction

An amount of money X of the medical expense deduction by the final tax return is calculated by the following formula. However, it is assumed that the upper limit of the amount of money X is two million Japanese Yen.

X=(S−Q)−R (1)

In the formula (1), “S” indicates a total amount of actual paid medical expenses, “Q” indicates an amount of money as compensation by insurance money and the like, and “R” is 100 thousand Japanese Yen. However, when a total income amount or the like of the year is less than two million Japanese Yen, “R” becomes an amount of money equivalent to 5% of the total income amount or the like.

When it is known from the personal information registered in the PDS system 303 or declaration at the time of user registration for the final tax return service that the Suzuki family's total income amount is two million Japanese Yen or more, “R” is hundred thousand Japanese Yen. In this case, “5” is a total amount of money of the item of “paid medical expense and transportation expense” in FIG. 10, which is 130,000 Japanese Yen. “Q” is a total amount of money of the item of “amount of money as compensation for the paid medical expense and transportation expense”, which is 0 Japanese Yen. Thus, X is calculated as follows.

X=(130000−0)−100000=30,000 (¥) (2)

In this case, “X” is a positive value, and therefore, the user may apply for the medical expense deduction.

### (C2) Calculation of a Self-Medication Tax System Deduction

The user is eligible for a deduction through the self-medication tax system as long as the user undergoes a specific regular medical checkup or the like. An amount of money Y of deductions by the self-medication tax system is calculated by the following formula. However, it is assumed that the upper limit of the amount of money Y is to 88,000 Japanese Yen.

Y=Z−12,000 (¥) (3)

In the formula (3), “Z” indicates an actual paid expense of medicine that is a self-medication target. When it is known from personal information registered in the PDS system 303 or declaration at the time of user registration for the final tax return service that Suzuki family undergoes a specific regular medical checkup or the like, the above-described precondition for the user is satisfied. In this case, “Z” is a total amount of money of the item of “price” in FIG. 11, which is 2,270 Japanese Yen. Thus, “Y” is calculated as follows.

Y=2270−12000=−9730<0 (¥) (4)

In this case, “Y” is a negative value, and therefore, the user is not allowed to apply for the self-medication tax system deduction.

Based on the calculation results of C1 and C2, application of a medical expense deduction may be proposed for Suzuki family. Therefore, the analysis circuit 348 generates analysis data that includes the data for the medical expense deduction in FIG. 10 and the analysis result usable for proposing application of the medical expense deduction, and the interface 347 notifies Taro Suzuki of the analysis data as application information.

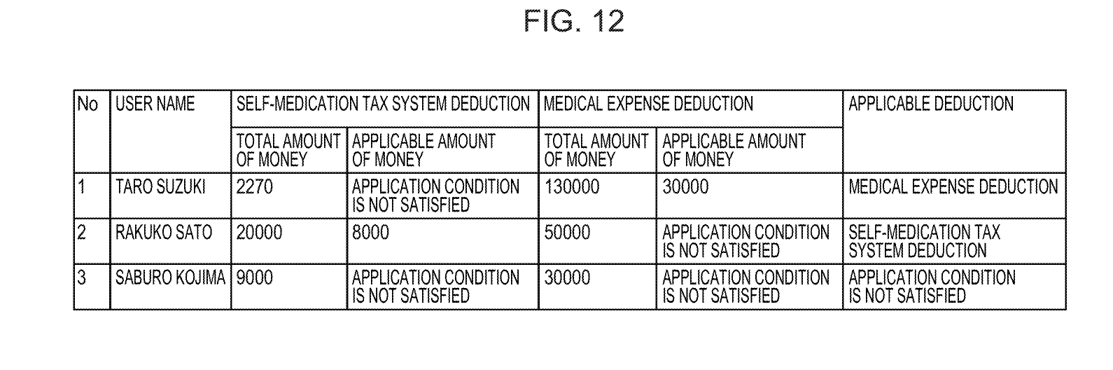

FIG. 12 illustrates an example of an analysis result included in analysis data stored in the analysis data DB 355. The analysis result in FIG. 12 includes items such as No, user name, self-medication tax system deduction, medical expense deduction, and applicable deduction.

“No” indicates an entry number, and “user name” indicates the name of the user. “Self-medication tax system deduction” indicates a calculation result of the self-medication tax system deduction, and includes items of “total amount of money” and “applicable amount of money”. “Total amount of money” indicates a sum of prices of self-medication target medicine purchased by the use, and “applicable amount of money” indicates an amount of money of deductions, which is calculated by the formula (3).

“Medical expense deduction” indicates a calculation result of the medical expense deduction and includes items of “total amount of money” and “applicable amount of money”. “Total amount of money” indicates a sum of medical expenses and transportation expenses that have been paid by the user, and “applicable amount of money” indicates an amount of money of deductions, which is calculated by the formula (1). “Applicable deduction” indicates an applicable deduction that is one of the self-medication tax system deduction and the medical expense deduction.

For example, an entry of No. 1 indicates an analysis result for the data of Suzuki family in FIGS. 10 and 11, and a total amount of money of deductions by the self-medication tax system is 2,270 Japanese Yen. In this case, the amount of money of deductions, which is calculated by the formula (3) is a negative value, and therefore, “Application condition is not satisfied” is recorded in the item of “applicable amount of money”.

On the other hand, the total amount of money of the medical expense deduction is 130,000 Japanese Yen, and the applicable amount of money is 30,000 Japanese Yen. Thus, the “applicable deduction” is a medical expense deduction. Taro Suzuki is notified of the data of the entry of No. 1 as application information.

The entries of Nos. 2 and 3 indicate analysis results of data of other users. The “applicable deduction” of the entry of No. 2 is the self-medication tax system deduction. In the item of the applicable deduction of the entry of No. 3, “application condition is not satisfied” is recorded, which indicates that the user is not allowed to apply for either of the self-medication tax system deduction or the medical expense deduction.

As described above, the application information of which the user is notified includes a deduction to be applied from among the self-medication tax system deduction and the medical expense deduction and a calculation result of an amount of money of the applicable deduction. Therefore, the user may easily recognize an appropriate deduction from among the self-medication tax system deduction or the medical expense deduction without complicated calculation for these deductions.

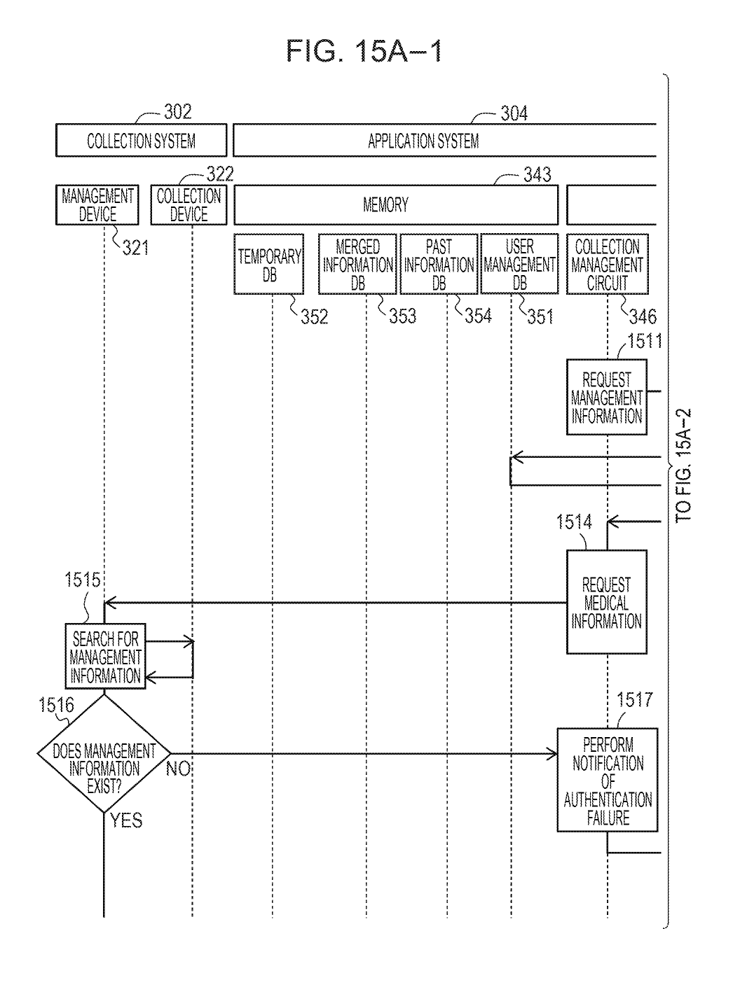

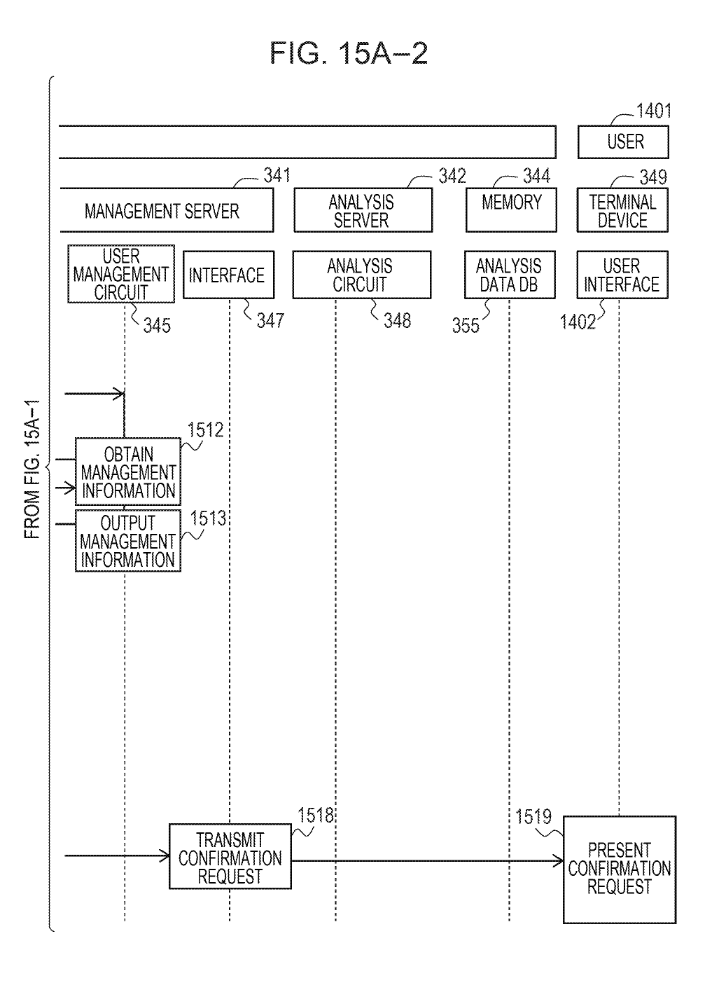

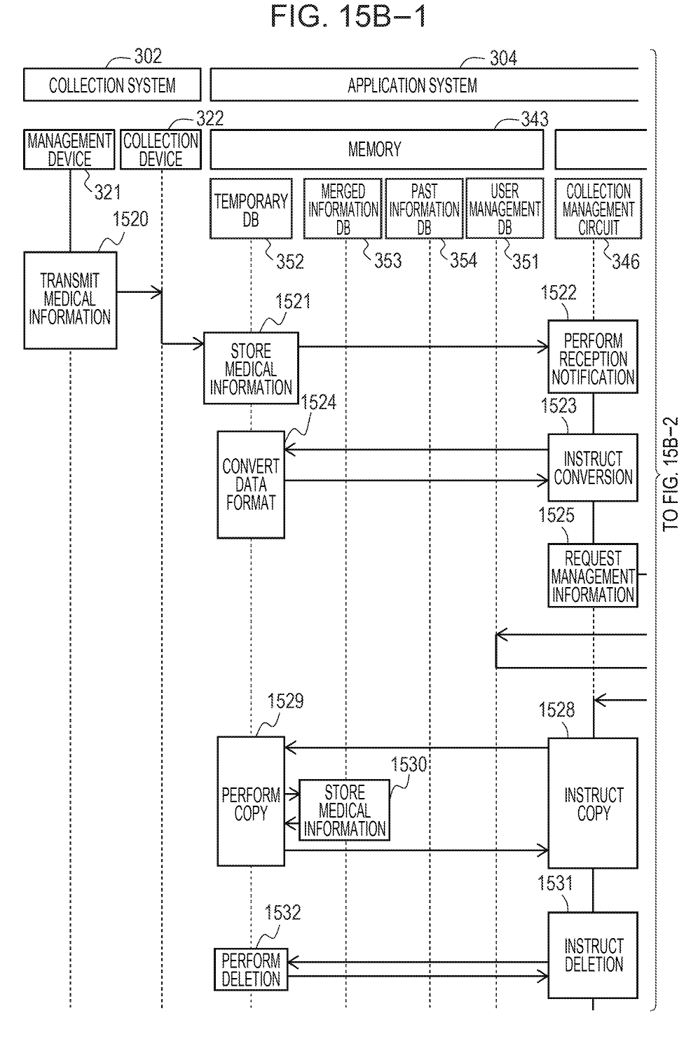

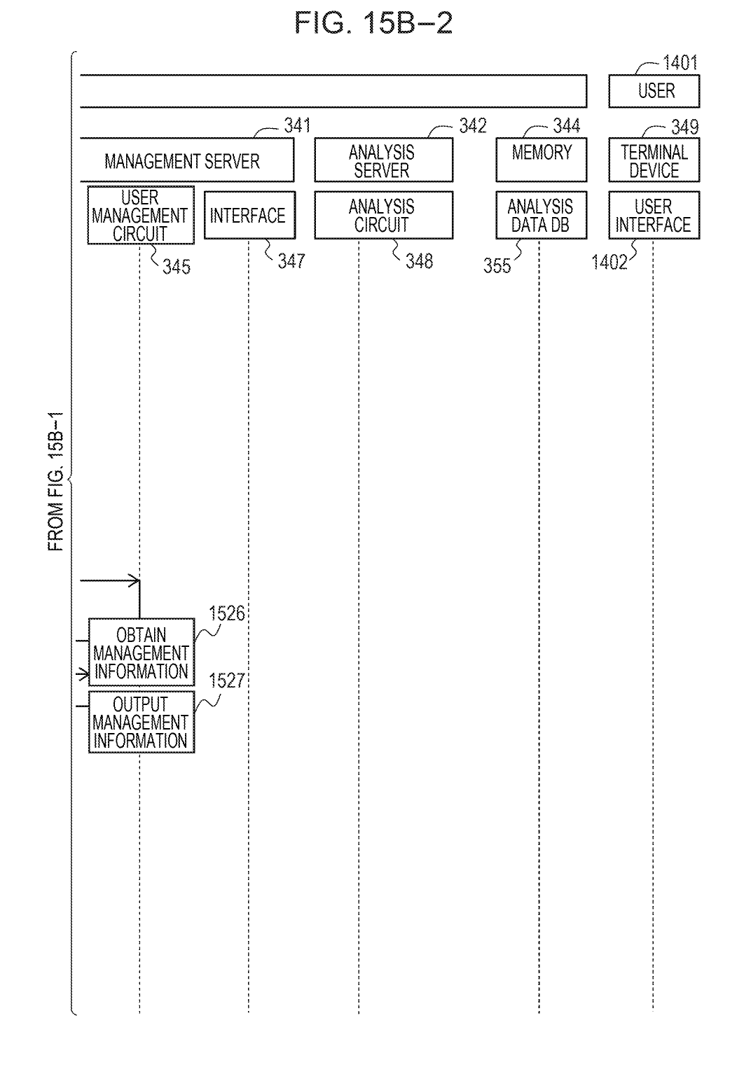

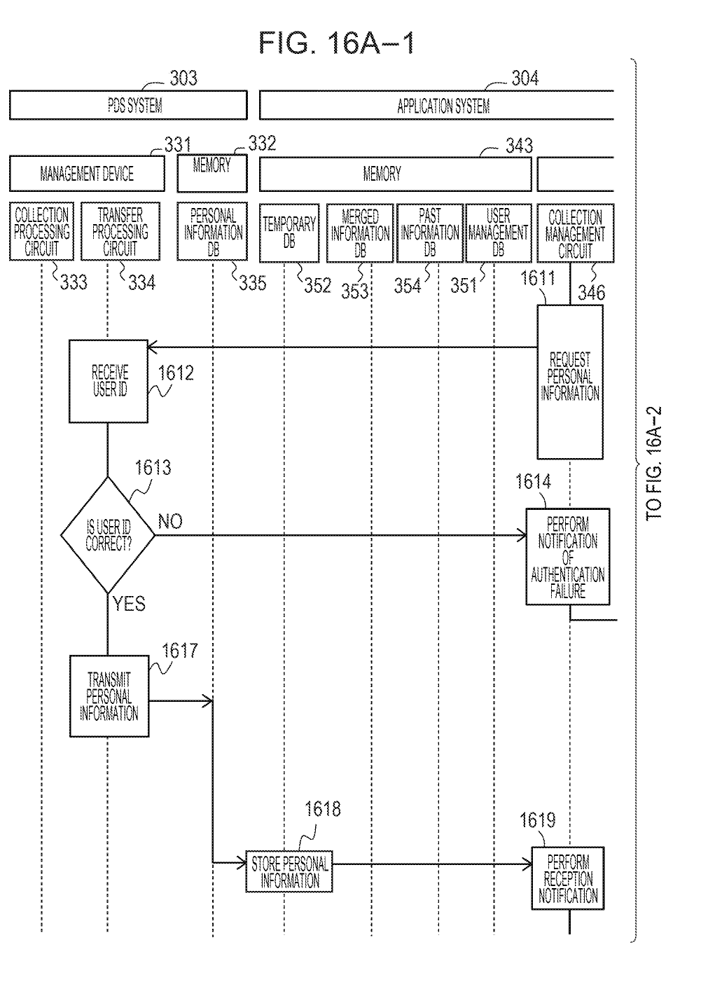

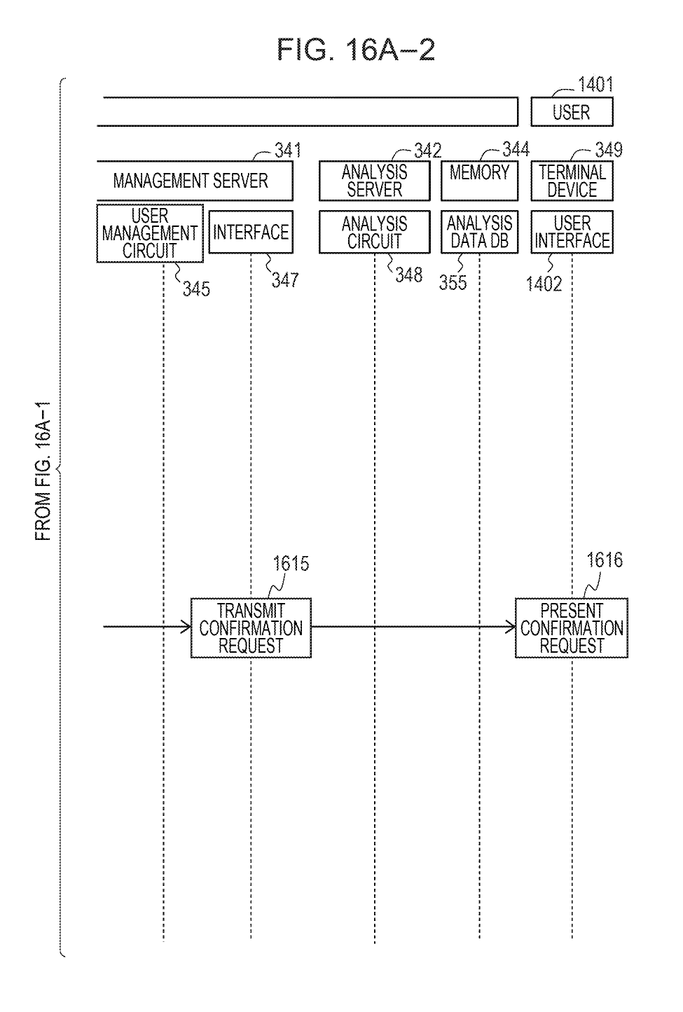

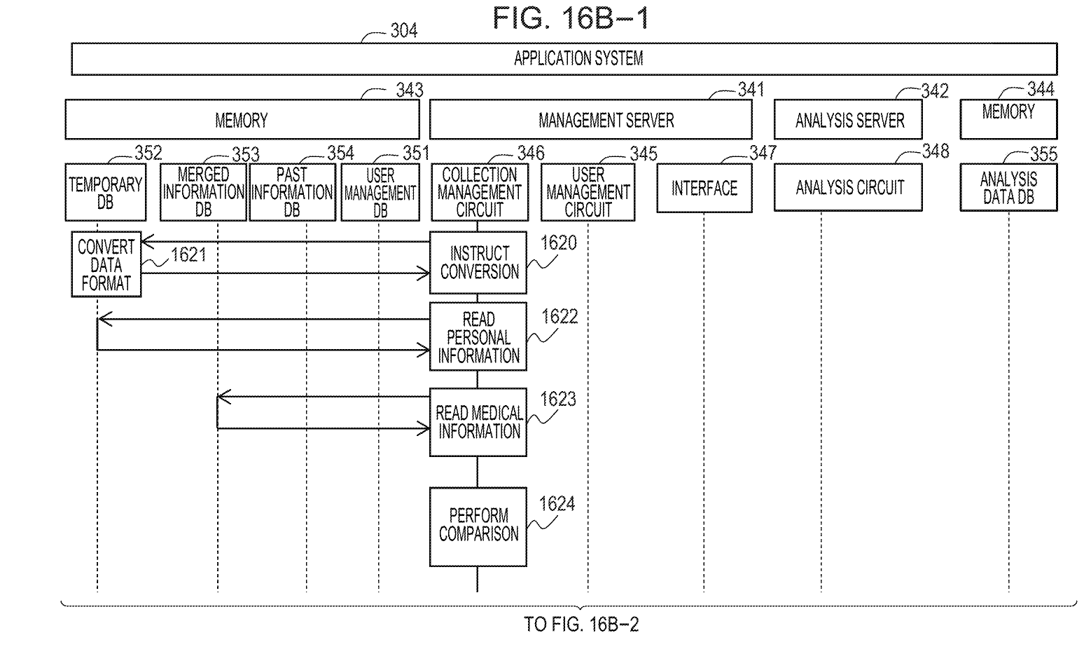

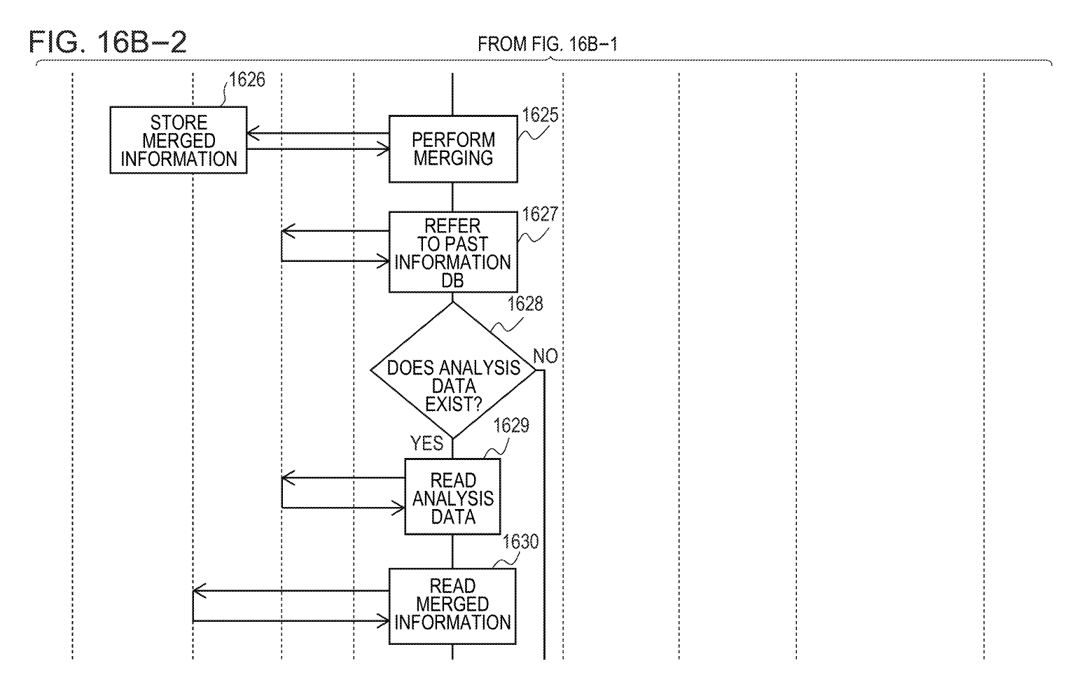

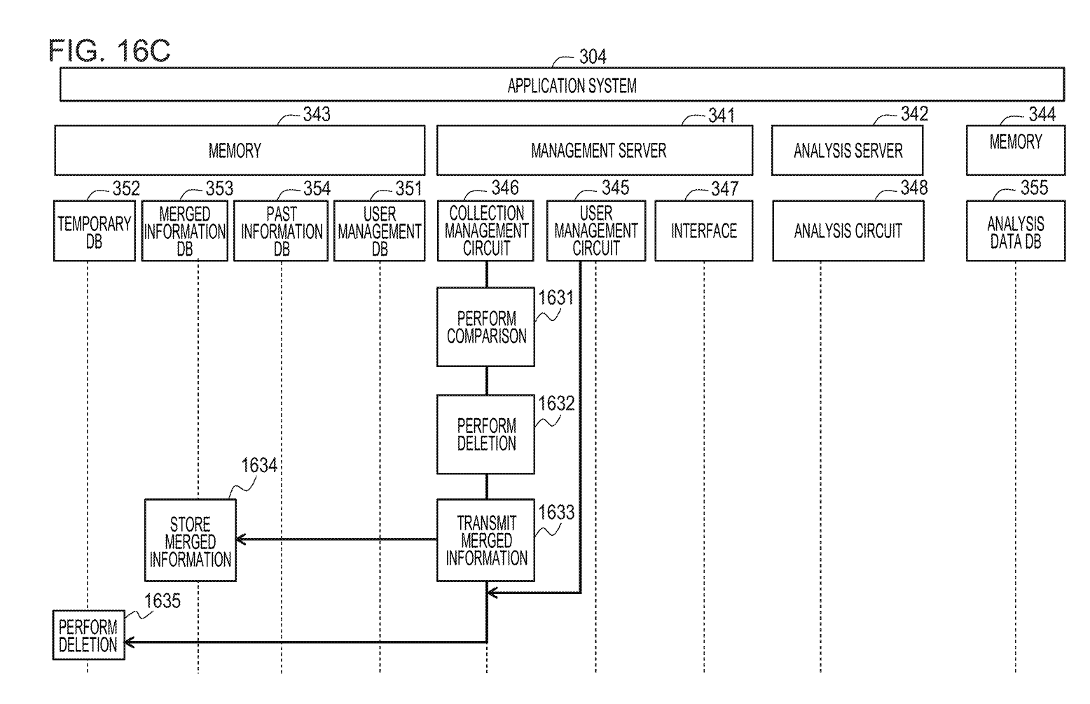

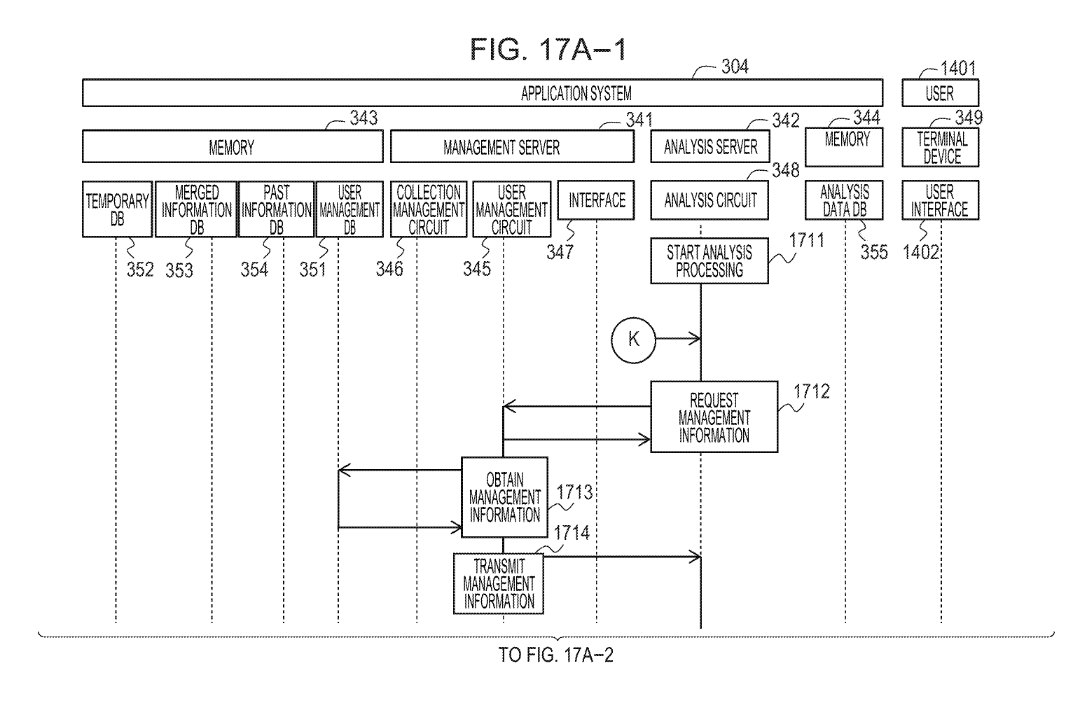

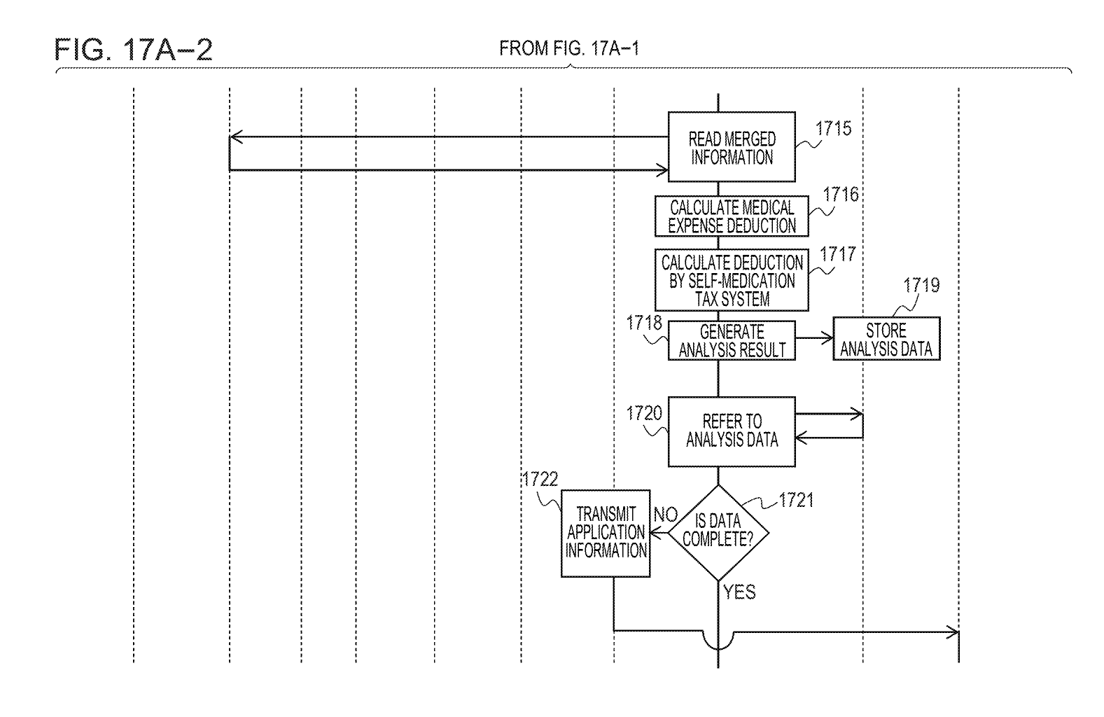

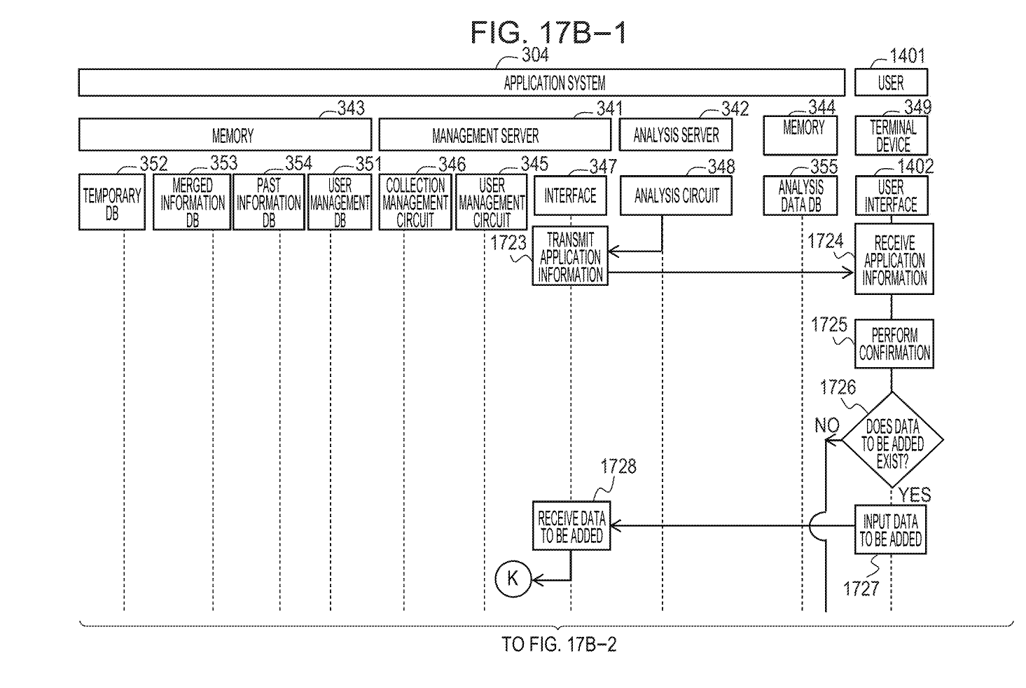

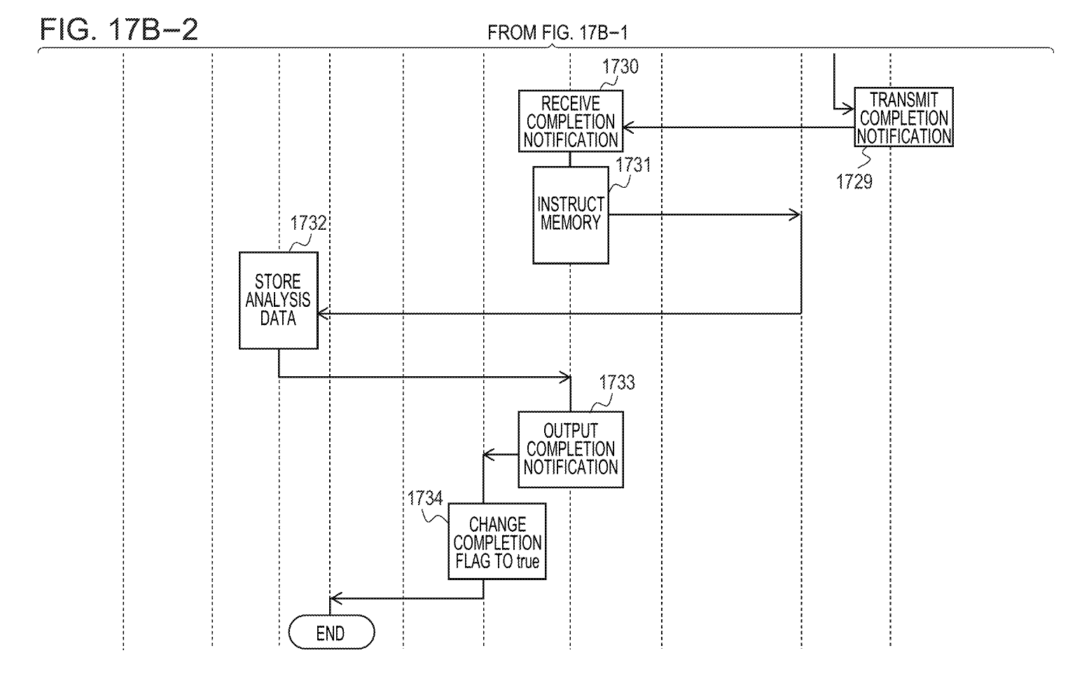

Details of an operation of the information processing system in FIG. 3 are described blow with reference to FIGS. 13 to 17B-2.

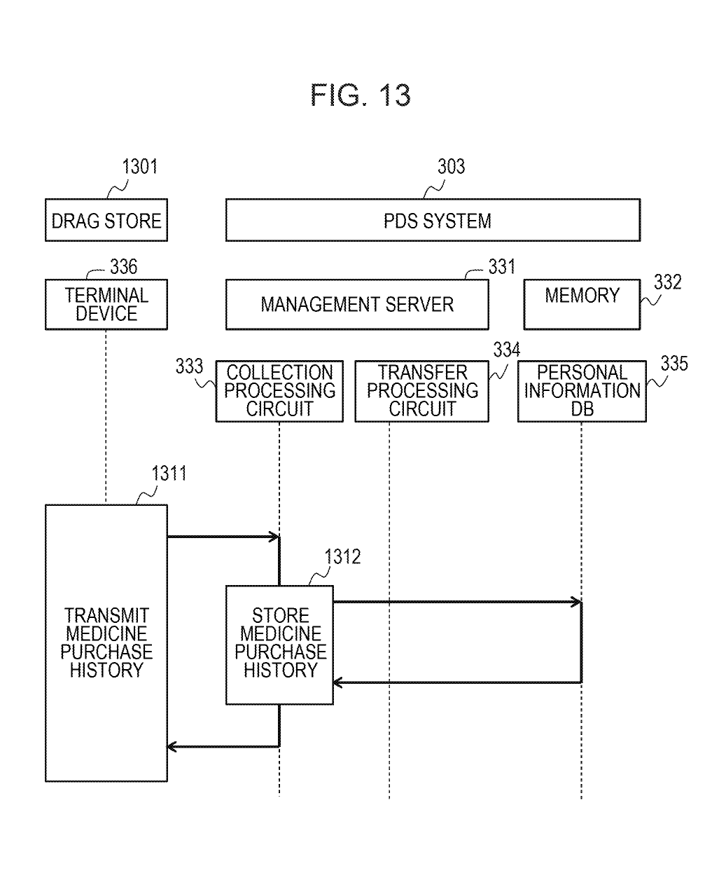

FIG. 13 illustrates an example of a registration sequence of registering personal information of the user in the PDS system 303. First, when the user purchases medicine in a drug store 1301, the terminal device 336 transmits medicine purchase history information to the management server 331 of the PDS system 303 and requests the management server 331 to store the information (step 1311). The collection processing circuit 333 of the management server 331 stores the received the medicine purchase history information in the personal information DB 335 of the memory 332 (step 1312).

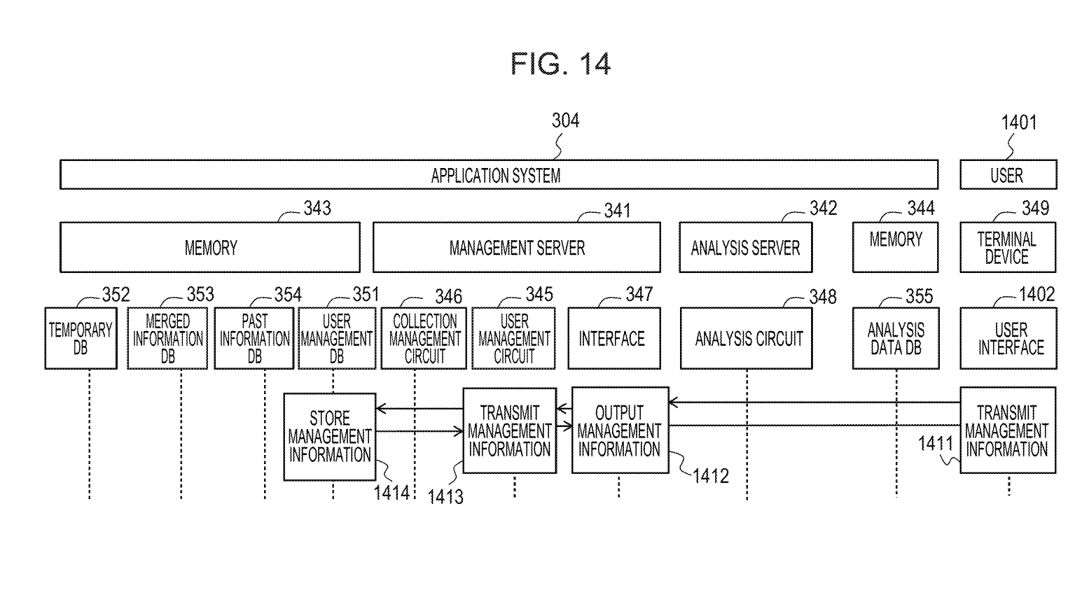

FIG. 14 illustrates an example of a registration sequence of registering management information of the user in the application system 304. In the terminal device 349 of a user 1401, a user interface 1402 such as a graphical user interface (GUI) or a command line interface (CLI) is installed. The user interface 1402 may be a browser.

First, the user interface 1402 transmits, to the management server 341, management information that the user has input to the terminal device 349 and requests the management server 341 to register the management information (step 1411). The interface 347 of the management server 341 outputs the received management information to the user management circuit 345 (step 1412).

Next, the user management circuit 345 transfers the management information to the memory 343 (step 1413), and the memory 343 stores the received management information in the user management DB 351 (step 1414).

FIGS. 15A-1, 15A-2, 15B-1 and 15B-2 illustrate an example of a collection sequence of collecting medical information of the user from the collection system 302. First, the collection management circuit 346 of the management server 341 requests the user management circuit 345 to provide management information of the user whose medical information is to be collected (step 1511). The user management circuit 345 obtains the requested management information of the user from the user management DB 351 (step 1512) and outputs the obtained management information to the collection management circuit 346 (step 1513).

Next, the collection management circuit 346 transmits the user ID in the collection system 302, which is included in the management information of the user, to the collection system 302 and requests the collection system 302 to provide medical information of the user, which is used for the final tax return (step 1514). The management device 321 of the collection system 302 searches the collection device 322 for management information (step 1515) and checks whether management information corresponding to the user ID exists (step 1516).

When management information corresponding to the user ID does not exist (step 1516, NO), the management device 321 notifies the collection management circuit 346 of the authentication failure (step 1517). The collection management circuit 346 transmits, to the terminal device 349, a confirmation request that requests the terminal device 349 to confirm management information of the user through the interface 347 (step 1518), and the user interface 1402 presents the received confirmation request to the user (step 1519).

When management information corresponding to the user ID exists (step 1516, YES), the management device 321 obtains an electronic medical record corresponding to the user ID from the collection device 322 and transmits medical information included in the obtained electronic medical record to the memory 343 (step 1520). At this time, the management device 321 extracts only medical information that conforms to a target time period and a target item of the final tax return and transmits the extracted medical information to the memory 343. The memory 343 stores the received medical information in the temporary DB 352 (step 1521).

The collection management circuit 346 receives a reception notification of the medical information from the memory 343 (step 1522) and instructs the memory 343 to convert the data format (step 1523). The memory 343 converts the medical information in the temporary DB 352 into the data format usable for the declaration of the final tax return (step 1524).

Next, the collection management circuit 346 requests the user management circuit 345 to provide management information corresponding to the user ID (step 1525). The user management circuit 345 obtains the requested management information of the user from the user management DB 351 (step 1526) and outputs the obtained management information to the collection management circuit 346 (step 1527).

Next, the collection management circuit 346 transmits, to the memory 343, the user ID for the final tax return service, which is included in the management information of the user, and instructs the memory 343 to copy the medical information (step 1528). The memory 343 copies the medical information in the temporary DB 352 to the merged information DB 353 (step 1529), associates the copied medical information with the user ID for the final tax return service, and stores the associated medical information and the user ID in the merged information DB 353 (step 1530).

Next, the collection management circuit 346 instructs the memory 343 to delete the medical information (step 1531), and the memory 343 deletes the medical information in the temporary DB 352 (step 1532).

FIGS. 16A-1, 16A-2, 16B-1, 16B-2 and 16C illustrate an example of a collection sequence of collecting personal information of the user from the PDS system 303. First, the collection management circuit 346 of the management server 341 transmits the user ID in the PDS system 303 to the PDS system 303 and requests to the PDS system 303 to provide personal information of the user, which is used for the final tax return (step 1611). The transfer processing circuit 334 of the PDS system 303 receives the user ID (step 1612) and checks whether the received user ID is correct (step 1613).

When the user ID is not correct (step 1613, NO), the transfer processing circuit 334 notifies the collection management circuit 346 of the authentication failure (step 1614). The collection management circuit 346 transmits a confirmation request that requests the terminal device 349 to confirm management information of the user, to the terminal device 349 through the interface 347 (step 1615), and the user interface 1402 presents the received confirmation request to the user (step 1616).

When the user ID is correct (step 1613, YES), the transfer processing circuit 334 obtains personal information corresponding to the user ID from the personal information DB 335 and transmits the obtained personal information to the memory 343 (step 1617).

At this time, the transfer processing circuit 334 extracts only personal information that conforms to target items of the final tax return and transmits the extracted personal information to the memory 343. For example, in a case of the personal information 401 illustrated in FIG. 4, the transfer processing circuit 334 extracts only an entry in which the “purpose” is “medical”, and in a case of the personal information 501 illustrated in FIG. 5, the transfer processing circuit 334 extracts only an entry in which the “self-medication target” is “◯”.

The memory 343 stores the received personal information in the temporary DB 352 (step 1618).

The collection management circuit 346 receives a reception notification of the personal information from the memory 343 (step 1619) and instructs the memory 343 to convert the data format (step 1620). The memory 343 converts the personal information in the temporary DB 352 into the data format usable for the declaration of the final tax return (step 1621).

Next, the collection management circuit 346 reads personal information from the temporary DB 352 (step 1622), reads medical information from the merged information DB 353 (step 1623), and compares the personal information with the medical information (step 1624). Next, the collection management circuit 346 extracts only entries of the personal information in the temporary DB 352, which do not duplicate entries of the medical information, and merges the extracted entry of the personal information with the medical information to generate merged information (step 1625). The collection management circuit 346 stores the generated merged information in the merged information DB 353 (step 1626).

Next, the collection management circuit 346 refers to the past information DB 354 (step 1627) and checks whether analysis data for the past five years exists (step 1628). When analysis data for the past five years exists (step 1628, YES), the collection management circuit 346 reads the analysis data for the past five years from the past information DB 354 (step 1629) and reads merged information from the merged information DB 353 (step 1630).

Next, the collection management circuit 346 compares merged information included in the analysis data for the past five years with the merged information read from the merged information DB 353 (step 1631). The collection management circuit 346 deletes information included in the analysis data from the merged information read from the merged information DB 353 (step 1632).

Next, the collection management circuit 346 transmits, to the memory 343, the merged information the part of which has been deleted (step 1633), and the memory 343 stores the received merged information in the merged information DB 353 (step 1634). The collection management circuit 346 deletes the personal information in the temporary DB 352 (step 1635). When analysis data for the past five years does not exist (step 1628, NO), the collection management circuit 346 executes the processing of step 1635.

FIGS. 17A-1, 17A-2, 17B-1 and 17B-2 illustrate an example of a generation sequence of generating application information from merged information. The analysis circuit 348 of the analysis server 342 starts analysis processing for merged information stored in the merged information DB 353 (step 1711). As the timing at which the analysis processing starts, for example, the following timings are assumed.

(a) Timing at which a request from the user is received
(b) Periodically (every month, every week, or the like)
(c) During a time from the beginning of the year to the final tax return declaration start
(d) Immediately after the collection sequence of collecting personal information

Next, the analysis circuit 348 requests the user management circuit 345 of the management server 341 to provide management information of the user, which is an analysis target (step 1712). The user management circuit 345 obtains the requested management information of the user from the user management DB 351 (step 1713) and transmits the obtained management information to the analysis circuit 348 (step 1714).

Next, the analysis circuit 348 reads merged information corresponding to the user ID as analysis target data from the merged information DB 353 (step 1715). The analysis circuit 348 calculates a medical expense deduction (step 1716) and calculates a self-medication tax system deduction (step 1717).

Next, the analysis circuit 348 generates an analysis result based on the calculation results of the medical expense deduction and the self-medication tax system deduction (step 1718). The analysis circuit 348 associates analysis data including the analysis target data and the analysis results with the user ID for the final tax return service and stores the analysis data and the user ID in the analysis data DB 355 of the memory 344 (step 1719).

Next, the analysis circuit 348 refers to analysis data stored in the analysis data DB 355 (step 1720) and checks whether data desired for the final tax return is complete (step 1721). As data desired for the final tax return based on the current law, for example, there is the following data.

### (I1) Income Amount

A calculation formula of a medical expense deduction changes depending on an income amount, and therefore, data of the income amount is desired for calculation of an amount of money of the medical expense deduction. For example, the collection management circuit 346 may collect an income amount of the user from tax withholding data registered in the PDS system 303.

### (I2) Certificate Document Defined by the Self-Medication Tax System

The self-medication tax system is a tax system in which the user who appropriately undergoes a medical checkup or the like may receive an income deduction when the user has purchased specific medicine. The user is obliged to submit a certificate document that proves that the user is actually undertaking a specific approach to “maintenance and improvement of health and prevention of diseases”. As the specific approach, for example, there are the following approaches.

(E1) Special health checkup (health check-up for metabolic syndrome)
(E2) Immunization (influenza or the like)
(E3) Regular medical checkup of company
(E4) Other health checkup such as complete medical checkup
(E5) Municipal cancer checkup

Thus, a certificate document such as a receipt, a result notification, or the like related to one of the approaches is desired for reception of a self-medication tax system deduction. The collection management circuit 346 may collect data on a certificate document of the user, for example, from medical checkup data registered in the PDS system 303.

Whether data desired for the final tax return is registered in the PDS system 303 depends on each user, and therefore, such data may not be registered in the PDS system 303.

When data desired for the final tax return is not complete (step 1721, NO), the interface 347 transmits analysis data as application information to the terminal device 349 and requests the user to provide missing data (step 1722). The terminal device 349 receives the application information (step 1724), and the user confirms the received application information (step 1725) and checks whether data to be added exists (step 1726). The data to be added includes data desired for the final tax return.

When data desired for the final tax return is complete (step 1721, YES), the interface 347 transmits analysis data as application information to the terminal device 349 (step 1723), and the terminal device 349 executes processing in step 1724 and subsequent steps.

When data to be added exists (step 1726, YES), the user inputs the data to be added, to the terminal device 349 through the user interface 1402, and the user interface 1402 transmits, to the management server 341, the data to be added (step 1727). The interface 347 receives the data to be added (step 1728), and the management server 341 and the analysis server 342 repeats the processing in step 1712 and subsequent steps. At this time, the collection management circuit 346 transfers the data to be added, which has been received from the terminal device 349, to the analysis server 342, and the analysis circuit 348 executes analysis processing by adding, to the analysis target data, the received data to be added.

When data to be added does not exist (step 1726, NO), the user inputs a notification indicating that data to be added does not exist, to the terminal device 349 through the user interface 1402. The user interface 1402 transmits, to the management server 341, a completion notification indicating that generation of application information has been completed (step 1729). The interface 347 receives the completion notification (step 1730) and instructs the memory 344 to store the analysis data through the analysis server 342 (step 1731).

The memory 344 transfers the analysis data stored in the analysis data DB 355 to the memory 343 through the analysis server 342 and the management server 341, and the memory 343 stores the received analysis data in the past information DB 354 (step 1732).

Next, the interface 347 outputs, to the user management circuit 345, a completion notification indicating that generation of the application information has been completed (step 1733). The user management circuit 345 changes a completion flag of the management information corresponding to the user ID in the user management DB 351 from “false” to “true” (step 1734).

The configuration of the information processing system illustrated in FIGS. 1 and 3 is only an example, and some of the configuration elements of the information processing system may be omitted or changed depending on a usage or a condition of the information processing system. For example, in the information processing system illustrated in FIG. 3, when the analysis circuit 348 is provided in the management server 341, the analysis server 342 may be omitted, and when the analysis data DB 355 is provided in the memory 343, the memory 344 may be omitted. The number of PDS systems 303 is not limited to one, and two or more PDS systems may be provided depending on the type of registered personal information.

As the hospital systems 301-1 and 301-2 in FIG. 3, a hospital system described in the prior Japanese Patent Application No. 2017-81245 may be used. In addition, as the collection system 302, a management system in an information management organization and a collection system in a data collection organization described in Japanese Patent Application No. 2017-81245 may also be used.

The flowchart illustrated in FIG. 2 and the operation sequence in FIGS. 13 to 17B-2 are only examples, and a part of the processing of the information processing system may be omitted or changed depending on a configuration or a condition of the information processing system. For example, the order of the collection processing of collecting medical information from the collection system 302 and the collection processing of collecting personal information from the PDS system 303 may be changed.

The personal information and the medical information in FIGS. 4 to 8 and the management information in FIG. 9 are only examples, and some or all the items of the information may be omitted or changed depending on a usage or a condition of the information processing system. The data of FIGS. 10 and 11 and the analysis result of FIG. 12 are only examples, and the analysis result changes depending on a usage or a condition of the information processing system. The formulas (1) to (4) are only examples, and a calculation formula of an amount of money of deductions changes according to revision of a tax system.

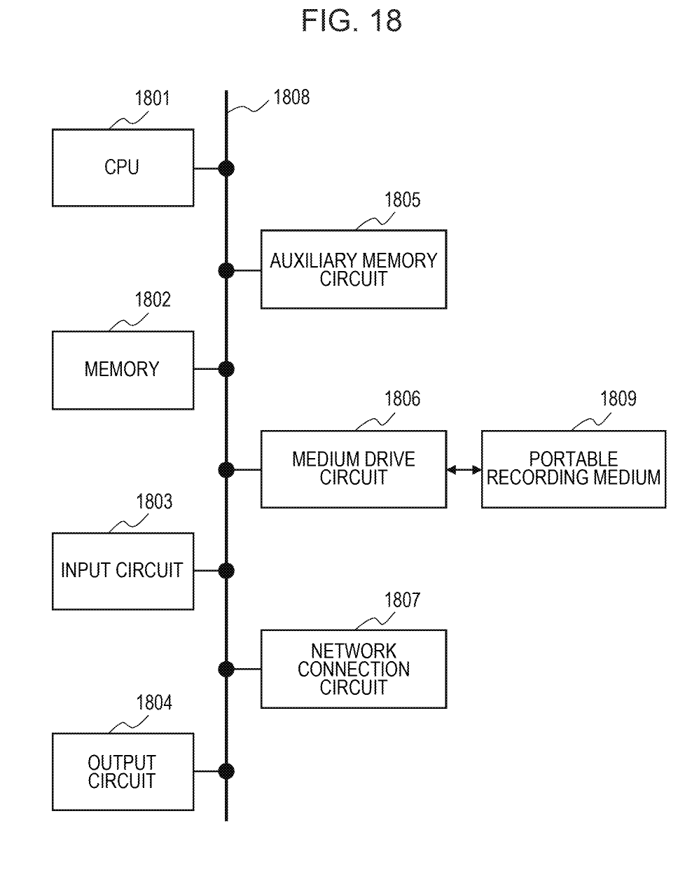

FIG. 18 illustrates a hardware configuration example of an information processing apparatus (computer) usable as the third information processing apparatus 103 in FIG. 1 and the management server 341 and the analysis server 342 in FIG. 3. The information processing apparatus in FIG. 18 includes a central processing unit (CPU) 1801, a memory 1802, an input circuit 1803, an output circuit 1804, an auxiliary memory circuit 1805, a medium drive circuit 1806, and a network connection circuit 1807. These configuration elements are coupled to each other through a bus 1808.

The memory 1802 is, for example, a semiconductor memory such as a read only memory (ROM), a random access memory (RAM), or a flash memory, and stores programs and data usable for processing.

The CPU 1801 (processor) operates, for example, as the generation circuit 112 in FIG. 1, the user management circuit 345, the collection management circuit 346, and the analysis circuit 348 in FIG. 3 by executing programs by using the memory 1802.

The input circuit 1803 includes, for example, a keyboard, and a pointing device, and is used for input of instruction or information from the operator. The output circuit 1804 includes, for example, a display device, a printer, and a speaker, and is used for output of an inquiry or a processing result to the operator.

The auxiliary memory circuit 1805 is, for example, a magnetic disk device, an optical disk device, a magneto optical disk device, a tape device, or the like. The auxiliary memory circuit 1805 may be a hard disk drive. The information processing apparatus stores programs and data in the auxiliary memory circuit 1805 and uses the programs and data by loading the programs and data into the memory 1802.

The medium drive circuit 1806 drives a portable recording medium 1809 to access a record content. The portable recording medium 1809 is a memory device, a flexible disk, an optical disk, a magneto optical disk, or the like. The portable recording medium 1809 may be a digital versatile disk (DVD), a compact disk read only memory (CD-ROM), a universal serial bus (USB) memory, or the like. The operator may store programs and data in the portable recording medium 1809 and use the programs and data by loading the programs and data into the memory 1802.

As described above, the computer-readable storage medium that stores programs and data usable for the processing is a physical (non-transitory) recording medium such as the memory 1802, the auxiliary memory circuit 1805, or the portable recording medium 1809.

The network connection circuit 1807 is a communication interface circuit coupled to a communication network such as a local area network (LAN) or a wide area network (WAN) and performs data conversion that accompanies with communication. The information processing apparatus may receive programs and data from an external device through the network connection circuit 1807 and use the programs and data by loading the programs and data into the memory 1802. The network connection circuit 1807 may be used as the communication circuit 111 in FIG. 1 and the interface 347 in FIG. 3.

The information processing apparatus may not include all the configuration elements in FIG. 18, and some of the configuration elements may be omitted depending on a usage or a condition. For example, when it is unnecessary to receive an input of instruction or information from the operator, the input circuit 1803 may be omitted, and when it is unnecessary to output an inquiry or a processing result to the operators, the output circuit 1804 may be omitted. When the portable recording medium 1809 is not used, the medium drive circuit 1806 may be omitted.

As the first information processing apparatus 101 and the second information processing apparatus 102 in FIG. 1, an information processing apparatus similar to that of FIG. 18 may be used. Even as the terminal device 311, the management server 312, the memory 313, the management device 321, the collection device 322, the management server 331, the memory 332, and the terminal device 336 in FIG. 3, the information processing apparatus similar to that of FIG. 18 may be used. Even as the memory 343, the memory 344, and the terminal device 349 in FIG. 3, the information processing apparatus similar to that of FIG. 18 may be used.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. An information processing system comprising:
a first information processing apparatus including:
a first memory configured to store medical information associated with identification information, and
a first processor configured to transmit medical information;
a second information processing apparatus including:
a second memory configured to store personal information associated with identification information, and
a second processor configured to transmit personal information; and
a third information processing apparatus including:
a third processor configured to execute a process, the process including:
transmitting, to the first information processing apparatus, first identification information that identifies medical information usable for specific application,
receiving medical information corresponding to the first identification information from the first information processing apparatus,
transmitting, to the second information processing apparatus, second identification information that identifies personal information usable for the specific application,
receiving personal information corresponding to the second identification information from the second information processing apparatus, and
generating application information usable for the specific application, based on the medical information received from the first information processing apparatus and the personal information received from the second information processing apparatus.

2. The information processing system according to claim 1, wherein the third information processing apparatus is configured to:
convert the medical information received from the first information processing apparatus and the personal information received from the second information processing apparatus into a specific format,
generate merged information by merging the medical information having the specific format with the personal information having the specific format while detecting duplicate information in the medical information having the specific format and the personal information having the specific format, and
generate the application information by analyzing the merged information.

3. The information processing system according to claim 1, wherein
the specific application is a final tax return filed by an applicant to which the first identification information and the second identification information are given,
the medical information received from the first information processing apparatus includes information on a medical expense that the applicant pays for at a medical institution,
the third information processing apparatus requests the second information processing apparatus to provide medicine purchase history information corresponding to the second identification information,
the personal information received from the second information processing apparatus includes information indicating a price of medicine purchased by the applicant, and
the application information indicates a deduction to be applied from among a medical expense deduction and a deduction through a self-medication tax system.

4. The information processing system according to claim 3, wherein
the third information processing apparatus requests the second information processing apparatus to provide information on a transportation expense corresponding to the second identification information,
the personal information received from the second information processing apparatus includes information on a transportation expense that the applicant pays to receive a medical examination at the medical institution, and
the third information processing apparatus generates medical expense deduction information including the medical expense that the applicant pays for at the medical institution and the transportation expense that the applicant pays to receive the medical examination at the medical institution.

5. The information processing system according to claim 3, wherein the third information processing apparatus is configured to:
generate the application information in accordance with a declaration time period of the final tax return, and
transmit the application information to a terminal device of the applicant.

6. An information processing apparatus comprising:
a communication circuit configured to:
transmit first identification information that identifies medical information usable for a specific application to a first information processing apparatus that stores medical information associated with identification information,
receive medical information corresponding to the first identification information from the first information processing apparatus,
transmit second identification information that identifies personal information usable for the specific application to a second information processing apparatus that stores personal information associated with identification information, and
receive personal information corresponding to the second identification information from the second information processing apparatus; and
a generation circuit configured to generate application information usable for the specific application, based on the medical information received from the first information processing apparatus and the personal information received from the second information processing apparatus.

7. The information processing apparatus according to claim 6, wherein the generation circuit is configured to:
convert the medical information received from the first information processing apparatus and the personal information received from the second information processing apparatus into a specific format,
generate merged information by merging the medical information having the specific format with the personal information having the specific format while detecting duplicate information in the medical information having the specific format and the personal information having the specific format, and
generate the application information by analyzing the merged information.

8. The information processing apparatus according to claim 6, wherein
the specific application is a final tax return filed by an applicant to which the first identification information and the second identification information are given,
the medical information received from the first information processing apparatus includes information on a medical expense that the applicant pays for at a medical institution,
the communication circuit requests the second information processing apparatus to provide medicine purchase history information corresponding to the second identification information,
the personal information received from the second information processing apparatus includes information indicating a price of medicine purchased by the applicant, and
the application information indicates a deduction to be applied from among a medical expense deduction and a deduction through a self-medication tax system.

9. The information processing apparatus according to claim 8, wherein
the communication circuit requests the second information processing apparatus to provide information on a transportation expense corresponding to the second identification information,
the personal information received from the second information processing apparatus includes information on a transportation expense that the applicant pays to receive a medical examination at the medical institution, and
the generation circuit generates medical expense deduction information including the medical expense that the applicant pays for at the medical institution and the transportation expense that the applicant pays to receive the medical examination at the medical institution.

10. The information processing apparatus according to claim 8, wherein
the generation circuit generates the application information in accordance with a declaration time period of the final tax return, and the communication circuit transmits the application information to a terminal device of the applicant.

11. A non-transitory computer-readable recording medium having stored therein a program for causing a computer to execute a process, the process comprising:
transmitting, to a first information processing apparatus, first identification information that identifies medical information usable for specific application;
receiving medical information corresponding to the first identification information from the first information processing apparatus;
transmitting, to a second information processing apparatus, second identification information that identifies personal information usable for the specific application;
receiving personal information corresponding to the second identification information from the second information processing apparatus; and
generating application information usable for the specific application, based on the medical information received from the first information processing apparatus and the personal information received from the second information processing apparatus.

12. The recording medium according to claim 11, the process further comprising:
converting the medical information received from the first information processing apparatus and the personal information received from the second information processing apparatus into a specific format;
generating merged information by merging the medical information having the specific format with the personal information having the specific format while detecting duplicate information in the medical information having the specific format and the personal information having the specific format; and
generating the application information by analyzing the merged information.

13. The recording medium according to claim 11, wherein
the specific application is a final tax return filed by an applicant to which the first identification information and the second identification information are given,
the medical information received from the first information processing apparatus includes information on a medical expense that the applicant pays for at a medical institution,
the computer requests the second information processing apparatus to provide medicine purchase history information corresponding to the second identification information,
the personal information received from the second information processing apparatus includes information indicating a price of medicine purchased by the applicant, and
the application information indicates a deduction to be applied from among a medical expense deduction and a deduction through a self-medication tax system.

14. The recording medium according to claim 11, wherein
the computer requests the second information processing apparatus to provide information on a transportation expense corresponding to the second identification information,
the personal information received from the second information processing apparatus includes information on a transportation expense that the applicant pays to receive a medical examination at the medical institution, and
the computer generates medical expense deduction information including the medical expense that the applicant pays for at the medical institution and the transportation expense that the applicant pays to receive the medical examination at the medical institution.

15. The recording medium according to claim 11, wherein
the computer generates the application information in accordance with a declaration time period of the final tax return and transmits the application information to a terminal apparatus of the applicant.
